(19) United States
(12) Patent Application Publication
(10) Pub. No.: US 2022/0308049 A1
(43) Pub. Date: Sep. 29, 2022

(54) LATERAL FLOW TEST STRIPS WITH COMPETITIVE ASSAY CONTROL

(71) Applicant: ELLUME LIMITED, East Brisbane, Queensland (AU)

(72) Inventors: Christopher MILLER, East Brisbane, Queensland (AU); Scott FRY, East Brisbane, New South Wales (AU); Sean PARSONS, East Brisbane, New South Wales (AU); Harmony DOUWES, East Brisbane, New South Wales (AU)

(21) Appl. No.: 17/614,459

(22) PCT Filed: May 27, 2020
(86) PCT No.: PCT/AU2020/050530

§ 371 Date: Nov. 26, 2021

(30) Foreign Application Priority Data
May 27, 2019 (AU) 2019901798

(51) Int. Cl. G01N 33/543

(52) U.S. Cl. CPC G01N 33/54366

(57) ABSTRACT

The present disclosure relates to lateral flow test strips comprising an active control and diagnostic devices comprising same for making determinations about the presence or absence of one or more target analytes in a sample. For example, the present disclosure relates to lateral flow test strips comprising an active control which recognises a control analyte which is abundant in the biological sample being tested e.g., such as human serum albumin (HSA) in blood, and diagnostic devices comprising same. In some examples, the lateral flow test strips of the disclosure may comprise an active control and one or more internal controls.

### RELATED APPLICATION DATA

This application claims the right of priority to Australian Provisional Application No. 2019901798, filed 27 May 2019, the complete contents of which is incorporated by reference herein in its entirety.

### TECHNICAL FIELD

The present disclosure relates to lateral flow test strips comprising an active control and diagnostic devices comprising same for making determinations about the presence or absence of one or more target analytes in a sample. For example, the present disclosure relates to lateral flow test strips comprising an active control which recognises a control analyte which is abundant in the biological sample being tested e.g., such as human serum albumin (HSA) in blood, and diagnostic devices comprising same. In some examples, the lateral flow test strips of the disclosure may comprise an active control and one or more internal controls.

### BACKGROUND

Lateral Flow Assays (LFAs) have been in use in the in vitro diagnostics market for over 25 years and are widely regarded as inexpensive, easy to use, rapid and qualitative tests that can be used in point-of-care or field-based settings.

LFAs exploit the migration of a liquid sample along a porous membrane material such as nitrocellulose. Capture and detection of one or more target analytes takes place as the sample flows across discrete zones or lines immobilised with a capture reagent. Various capture reagents can be used, though antibodies are a popular choice. LFAs in which antibodies are used are typically referred to as Lateral Flow Immunoassays (LFIAs).

LFAs can be used for the detection of large complex analytes using a sandwich assay format or for the detection of small molecules or haptens using a competitive format. In a sandwich assay, typically a strip is assembled with a series of absorbent pad materials that direct the flow of sample and assay reagents across a series of discrete zones during which the target analyte is tagged (i.e. labelled) and subsequently captured and detected. The specimen is initially applied to an absorbent sample pad of the strip, which acts as a filter and a reservoir for the sample. Fluid is drawn, from the sample pad, through a conjugate release pad of the strip, where one or more target analytes in the sample are labelled by interacting with colorimetric, fluorescent, magnetic or radioactive reporter molecules. To effect the labelling, the reporter molecules are coupled to an analyte-specific ligand (usually an antibody), which rapidly forms complexes with respective target analytes to form labelled complexes. The sample, including labelled complexes contained therein, is drawn from the conjugate release pad to a test zone of the strip where one or more complementary ligands are immobilised onto the strip, at one or more test lines, to bind to the labelled complexes. The remaining sample continues its journey through the strip from the test zone to a highly absorbent sink pad. The presence of any labelled complexes at the one or more test zones provides a measurable indication of the presence of the one or more target analytes in the sample. Depending on the choice of label, the test may be interpreted by the naked eye, for example, whereby the presence of one or more ‘visible’ test lines provides a qualitative indication of the presence of one or more target analytes, or using a scanner e.g., which detects fluorescence.

LFA test strips also typically include an internal control to confirm successful performance of the test in the event that no analyte is detected in a sample. In traditional lateral flow assays, unbound labels flowing downstream of the test lines are captured by an anti-species (e.g. anti-mouse) antibody immobilised at a control line. The appearance of a control line provides evidence that the test has run properly acting as positive reinforcement for the user in case of a negative test outcome, where otherwise no band would appear. It also provides some indications that the biological components on the test trip remained active during transport and storage. In certain instances, for example when a test is destined for home use, a diagnostic test based on LFA could benefit from a more informative control to improve validation of test outcome. Accordingly, there is a need for LFA test strips with improved controls.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

In traditional lateral flow, unbound labels flowing downstream of the test lines are captured by an anti-species (e.g. anti-mouse) antibody at a control line. The appearance of a detectable signal at a control line provides evidence that the lateral flow test has run properly acting as positive reinforcement for the user in case of a negative test outcome, where otherwise no band would appear. The control also provides some indications that the biological components on the lateral flow test strip remained active during transport and storage. In certain instances, for example when a test is destined for home use, the test could benefit from a more active control. For example, instead of simply capturing unbound labels, an active control may specifically recognise a biomarker which is present in the biological sample.

The present inventors have developed a lateral flow test strip with an active control design based on a competition assay, which permits a user to more confidently determine whether a lateral flow assay is working correctly. In this regard, the present disclosure is based, in part, on the recognition that commercially available lateral flow tests include internal or active controls which are susceptible to the “hook effect” or “prozone effect” when a test analyte is present in intermediate to high concentrations. Traditional internal controls rely on unbound label flowing downstream of the test lines to be captured by an anti-species (e.g. anti-mouse) antibody at a control line. Similarly, traditional internal controls rely on a control analyte flowing downstream of the test lines to be captured by an appropriate antibody at a control line. In each case, the appearance of a detectable signal at a control line provides evidence that the lateral flow test has run properly acting as positive reinforcement for the user in case of a negative test outcome, where otherwise no band would appear. However, such controls may fail to provide an accurate evidence of lateral flow test performance when a test analyte is present in intermediate to high concentrations (due to “hooking out”), thereby leading a user to believe that the lateral flow test failed when in fact it did not.

The present disclosure provides lateral flow test strips and devices that include controls which are not susceptible to the “hook effect”. This is achieved by inclusion of an “active control” which relies on a competition assay to detect the presence or absence of a control analyte in a sample (or running buffer comprising same). A control design which includes an active control and which relies on a competition assay increases the dynamic range of the control and thereby permits more accurate validation of test results, particularly when the test analyte is present in an abundant amount.

Furthermore, the present inventors have developed a lateral flow test strip which comprises an “internal control”, in addition to the “active control” as described above. The inclusion of an internal control permits the user to confirm that a liquid sample to be tested (e.g., which may be a test sample alone or a mixture of running buffer and test sample) has travelled through the test strip during the lateral flow process and that all of the components of the test strip are performing as intended. Collectively, this dual control design enables a user to determine (i) whether or not sufficient control analyte (and thus sufficient test sample) is present during use (by virtue of the active control), (ii) that the test sample (or running buffer comprising same) has travelled through the test strip during the lateral flow process and that all of the components of the test strip are performing as intended, irrespective of whether the control analyte is present (by virtue of the internal control), and (iii) confirm the integrity of the assay reagents (which may be damaged or compromised due to exposure to humidity, light and/or oxygen (e.g., such as where package seal is broken) or which may have degraded over time (e.g., post expiry) which may result in a loss of functionality of the anti-species control. The internal control thereby acts as positive reinforcement for the user in the case of a positive test outcome.

Accordingly, in one aspect, the present disclosure provides a lateral flow test strip comprising:

a) a first mobilisable labelled species capable of binding to a first control analyte;

b) a first control portion comprising a first immobilised capture reagent;

wherein the first immobilised capture reagent mimics at least one binding property of the first control analyte such that the first immobilised capture reagent is capable of binding to the mobilisable labelled species.

In another aspect, the present disclosure provides a lateral flow test strip comprising:

a) a first mobilisable labelled species mimicking at least one binding property of a first control analyte;

b) a first control portion comprising a first immobilised capture reagent; wherein the first immobilised capture reagent is capable of binding to the mobilisable labelled species or to the first control analyte.

In one example, the first control analyte is human serum albumin (HSA). However, a skilled person will appreciate that the first control analyte may be any analyte which is present, preferably abundant, in a test sample. In accordance with an example in which the first control analyte is HSA and the first mobilisable labelled species binds thereof, the first mobilisable labelled species may be an anti-HSA antibody attached or conjugated to a detectable label, and the first immobilised capture reagent may be HSA. In accordance with another example in which the first control analyte is HSA and the first mobilisable labelled species mimics a binding property thereof, the first mobilisable labelled species may be HSA attached or conjugated to a detectable label, and the first immobilised capture reagent may be an antibody configured to bind HSA.

In accordance with aspects of the disclosure in which the lateral flow test strip comprises a single control portion i.e., a first control portion, the absence of, or a reduction in, detectable signal at the first control portion during use may be indicative that the lateral flow assay has been performed correctly. This is because, as the test sample flows through the test strip to the first test portion, the first control analyte comprised therein competitively binds to either the first mobilisable labelled species or the first immobilised capture reagent, as appropriate, thereby preventing or reducing binding of the first mobilisable labelled species to the first immobilised capture reagent. Conversely, where the first control analyte is not present or unable to bind the first immobilised capture reagent e.g., in circumstances where only running buffer has travelled through the lateral flow test strip or the control analyte is degraded, the first mobilisable labelled species will be free to bind to the first immobilised capture reagent during the lateral flow process. This will result in a detectable signal at the first control portion.

In some examples, the lateral flow test strip may further comprise:

c) a second mobilisable labelled species; and

d) a second control portion comprising a second immobilised capture reagent,

wherein the second immobilised capture reagent is capable of binding to the second mobilisable labelled species.

The second mobilisable labelled species may be a second control analyte which is attached or conjugated to a detectable label. In one example, the second mobilisable labelled species is chicken IgY attached or conjugated to a detectable label and the second immobilised capture reagent is configured to bind thereto e.g., an anti-species capture antibody raised against chicken IgY. However, a skilled person will appreciate that other immunoglobulins may be used as the second control analyte. Preferably the second control analyte is an immunoglobulin which is structurally different from mammalian IgG antibodies and has no cross-reactivity with known interferants e.g., in humans, such as complement, rheumatic factors or Fc receptors. The second control analyte may also be selected on the basis that anti-species capture antibodies raised against the second control analyte are commercially available. For example, in the case of chicken IgY, several anti-species capture antibodies raised against chicken IgY are commercially available e.g. goat anti-chicken IgY, donkey F(ab′)₂ anti-chicken IgY, rabbit F(ab′)₂ anti-chicken IgY and monoclonal mouse anti-chicken IgY.

In accordance with an example in which the second mobilisable labelled species is chicken IgY attached or conjugated to a detectable label, the second immobilised capture reagent may be an anti-chicken IgY antibody. For example, goat anti-chicken IgY, donkey F(ab′)₂ anti-chicken IgY, rabbit F(ab′)₂ anti-chicken IgY and monoclonal mouse anti-chicken IgY. However, where a different second control analyte is chosen, the second immobilised capture reagent will be configured to bind that particular analyte.

A lateral flow test strip of the disclosure comprising a second mobilisable labelled species and a second control portion as described herein provides a further level of certainty as to whether the lateral flow assay has been performed correctly. In use, the detection of signal at the second control portion may be indicative that the sample (optionally comprised in or comprising a running buffer) has travelled through the test strip during the lateral flow process, irrespective of whether the first control analyte is present. For example, a lateral flow test strip of the disclosure may be configured such that, in use:
(i) detection of a signal at the second control portion and no or reduced signal at the first control portion (wherein reduced signal is relative to the level of signal at the second control portion) is indicative that the lateral flow process proceeded correctly and the first control analyte was present in the sample (“pass”);
(ii) detection of a signal at the first control portion and at the second control portion is indicative that the lateral flow process proceeded correctly, but the first control analyte was not present in the sample (“fail”);
(iii) detection of a signal at the first control portion and not at the second control portion is indicative that the first control analyte was present in the sample but the lateral flow process did not proceed correctly e.g., the sample did not reach the second control portion and/or the second mobilisable labelled species or second immobilised capture reagent did not perform as intended (“fail”);
(iv) detection of no signal at the first or second control portions is indicative that the lateral flow process did not proceed correctly e.g., the first control analyte was not present in the sample and/or the sample did not reach the second control portion and/or the second mobilisable labelled species or second immobilised capture reagent did not perform as intended (“fail”).

The or each detectable label may be a latex particle, a nanoparticle aggregate, colloidal gold, a magnetic particle, fluorescent dye or quantum dot. In one example, the or each detectable label is a latex particle e.g., a glutaraldehyde-activated latex particle. In another example, the or each detectable label is a nanoparticle aggregate. In another example, the or each detectable label is colloidal gold. In another example, the or each detectable label is a magnetic particle. In another example, the or each detectable label is a fluorescent dye. In yet another example, the or each detectable label is a quantum dot.

In one example, the first and second mobilisable labelled species comprise the same detectable labels. In another example, the first and second mobilisable labelled species comprise different detectable labels.

In use, and in the absence of the first control analyte in a test sample, the first mobilisable labelled species binds to the first immobilised capture reagent. Binding of the first mobilisable labelled species to the first immobilised capture reagent will result in a detectable signal at the first control portion. This is indicative that the lateral flow assay has not proceeded correctly e.g., because (i) the first control analyte has degraded or (ii) there is insufficient or no test sample in the lateral flow running buffer applied to the test strip (e.g., the user has not applied sufficient test sample). However, when the first control analyte is present in a test sample, the first mobilisable labelled species binds to the first immobilised capture reagent at a reduced level compared to the level of binding (that would otherwise have occurred) in the absence of the first control analyte. This is because the first control analyte competitively binds to the first mobilisable labelled species, such that less or none of the first mobilisable labelled species is available for binding to the first immobilised capture reagent. This is indicative that the lateral flow assay has proceeded properly and that the test sample has flowed through the lateral flow test strip to the control portion.

In another aspect, the present disclosure provides a lateral flow test strip comprising:

a) a mobilisable labelled species which is bound to a first control analyte and a second control analyte;

b) a first control portion comprising a first immobilised capture reagent, wherein the first immobilised capture reagent is configured to specifically bind to the first control analyte;

c) and a second control portion comprising a second immobilised capture reagent, wherein the second immobilised capture reagent is configured to specifically bind to the second control analyte;

wherein the first control analyte is an analyte which is typically present in a test sample and wherein the second control analyte is an analyte which is not typically present in the test sample.

In use, and in the absence of the first control analyte in a test sample, the amount of the mobilisable labelled species immobilised at the first control portion is about equal to the amount of the mobilisable labelled species immobilised at the second control portion. For example, in the absence of the first control analyte in the test sample, the amount of mobilisable labelled species immobilised at the first control portion and the amount of mobilisable labelled species immobilised at the second control portion is present in about a 1:1 ratio to about 2:1 ratio. For example, in the absence of the first control analyte in the test sample, the amount of mobilisable labelled species immobilised at the first control portion and the amount of mobilisable labelled species immobilised at the second control portion is present in about a 1:1 ratio. For example, in the absence of the first control analyte in the test sample, the amount of mobilisable labelled species immobilised at the first control portion and the amount of mobilisable labelled species immobilised at the second control portion is present in about a 1.5:1 ratio. For example, in the absence of the first control analyte in the test sample, the amount of mobilisable labelled species immobilised at the first control portion and the amount of mobilisable labelled species immobilised at the second control portion is present in about a 2.1 ratio.

In use, and in the presence of the first control analyte in a test sample, the amount of the mobilisable labelled species immobilised at the first control portion is less than the amount of mobilisable labelled species immobilised at the second control portion. For example, in the presence of the first control analyte in the test sample, the amount of mobilisable labelled species immobilised at the first control portion and the amount of mobilisable labelled species immobilised at the second control portion is present in less than a 1:1 ratio.

The mobilisable labelled species bound to the first and second control analytes may be any labelled species e.g., a detectable label species. For example, the labelled species may be a latex particle, a nanoparticle aggregate, fluorescent dye or quantum dot . In one example, the labelled species is a latex particle e.g., a glutaraldehyde-activated latex particle. In another example, the labelled species is a nanoparticle aggregate. In another example, the labelled species is a fluorescent dye. In yet another example, the labelled species is a quantum dot.

In one example, the first control analyte is human serum albumin (HSA). However, a skilled person will appreciate that the first control analyte may be any analyte which is present, preferably abundant, in the test sample.

In one example, the second control analyte is chicken IgY. However, a skilled person will appreciate that other immunoglobulins may be used. Preferably the second control analyte is an immunoglobulin which is structurally different from mammalian IgG antibodies and has no cross-reactivity with known interferants e.g., in humans, such as complement, rheumatic factors or Fc receptors. The second control analyte may also be selected on the basis that anti-species capture antibodies raised against the second control analyte are commercially available. For example, in the case of chicken IgY, several anti-species capture antibodies raised against chicken IgY are commercially available e.g. goat anti-chicken IgY, donkey F(ab′)₂ anti-chicken IgY, rabbit F(ab′)₂ anti-chicken IgY and monoclonal mouse anti-chicken IgY.

In accordance with an example in which the first control analyte is HSA, the first immobilised capture reagent is an anti-human serum albumin antibody. However, where a different first control analyte is chosen, the first immobilised capture reagent will be configured to bind that particular analyte.

In accordance with an example in which the second control analyte is chicken IgY, the second immobilised capture reagent is an anti-chicken IgY antibody. For example, goat anti-chicken IgY, donkey F(ab′)₂ anti-chicken IgY, rabbit F(ab′)₂ anti-chicken IgY and monoclonal mouse anti-chicken IgY. However, where a different second control analyte is chosen, the second immobilised capture reagent will be configured to bind that particular analyte.

In accordance with any aspect of the present disclosure describing a lateral flow test strip which comprises two control portions, the first and second control portions may be configured such that the second control portion is positioned downstream of the first control portion, or vice versa.

In each of the foregoing aspects of the disclosure, the mobilisable labelled species may be located at one or more label-holding portions positioned upstream of the control portion(s). Alternatively, the mobilisable labelled species may be placed on the one or more label-holding portions e.g., using a sample dropper, prior to use. In yet another example, the mobilisable labelled species may be added to and mixed with the test sample prior to the test sample being applied to the test strip e.g., at the sample receiving portion thereof.

The lateral flow test strip of any aspect described herein may further comprise one or more test portions, each comprising an immobilised capture reagent configured to specifically bind to and thereby immobilise a test analyte to the test portion. The capture reagent of the or each test portion may be an antibody which is immobilised to the respective test portion of the lateral flow test strip. An appropriate antibody may be selected based on the test analyte to be immobilised.

The or each test portion may be positioned upstream of the or each control portion on the lateral flow test strip.

In some examples, labelling of the test analyte(s) may occur as part of the lateral flow process. For example, the lateral flow test strip may comprise one or more mobilisable capture reagents configured to bind to the test analyte(s) in the sample, wherein said mobilisable capture reagents comprise a detectable label. The mobilisable capture reagents configured to bind to the test analyte(s) may be positioned at a label holding portion of the test strip upstream of the respective test portion. In some examples, the detectably labelled mobilisable capture reagents configured to bind to the test analyte(s) may be positioned at the same label holding portion as the mobilisable labelled species. In use, labelled complexes which are formed between the test analyte and the mobilisable capture reagent during the lateral flow process can be immobilised at the respective test portion and detected by virtue of the detectable label.

In other examples, labelling of the test analyte(s) may occur separately to the lateral flow process. For example, the labelling of the test analyte(s) may occur upstream of the lateral flow process e.g., as part of an incubation step between the test sample (potentially comprising the test analyte) and a labelled mobilisable capture reagent configured to bind to the test analyte(s) as described herein. The test sample may be prepared in a solute form. Any labelled complexes formed between the test analyte and labelled mobilisable capture reagent may be distributed relatively uniformly throughout the test sample. A test sample comprising the labelled complexes may then be received at a sample receiving portion of the lateral flow test strip of the disclosure and travel therethrough under capillary action to reach the or each test portion during the lateral flow process. In accordance with this example, the lateral flow test strip needn't comprise the labelled mobilisable capture reagent configured to bind to the test analyte(s). The labelled mobilisable capture reagent may be provided separately.

As described herein, the lateral flow test strips of the disclosure may comprise a sample receiving portion configured to contact a test sample from a subject e.g., urine or blood, or a component part of the sample. The sample receiving portion may be upstream of the label-holding portion, the test portion and the or each control portion of the test strip.

The test sample with which the lateral flow test strip may be used may be any biological sample. In one example, the test sample is a human sample. In one example, the test sample is a mucus sample. In one example, the test sample is a blood sample or a component part thereof. In one example, the test sample is a urine sample.

In other examples the test sample may be obtained from a plant, animal or environmental source. In accordance with an example in which the test sample is obtained from an animal, the test sample may be a mucus sample, a blood sample or component part thereof or a urine sample. In accordance with an example in which the test sample is plant based, the test sample may be a plant tissue e.g., leaf, seed, fruit or roots, or one or more components obtained from the plant tissue e.g., oil, protein, DNA, RNA or combinations thereof. In accordance with an example in which the test sample is an environmental sample, the sample may be a water sample or an eluate obtained from a soil sample.

The present disclosure also provides an apparatus configured to receive the lateral flow test strip described herein and, during use, configured to present information to a user via a display relating to the identification of control analyte(s) at the respective control portion(s) and the identification of a test analyte in a test sample. The apparatus may be configured to allow removal of a used lateral flow test strip from its casing after use and subsequent replacement with a new test strip.

In one example, the apparatus is provided in the form of a hand-held device.

In one example, the apparatus may comprise a reader to identify control analyte(s) at the respective control portion(s) and test analyte at the test portion. For example, the reader may include one or more photodetectors capable of monitoring light reflection or light output at the control portion(s) and test portion.

In general, the signals at the control portion(s) and the test portion, which may be monitored or detected, may comprise light signals such as light reflection signals and/or fluorescent light signals or otherwise. The light signals may be generated as a result of detectable label immobilised at the first and/or second control portions and at the test portion which reflects light and/or which fluoresces light. The apparatus may comprise a light source that shines light on the test portion(s) and test portion to cause light reflection and/or fluorescing. Monitoring or detecting the presence and/or level of such light signals may comprise determining an absolute or relative intensity of the signals, for example. The absolute or relative intensity of the signals will be dependent on the number and type of detectable labels immobilised at the test portion(s) and test portion.

The present disclosure also provides a method of detecting a test analyte in a test sample by lateral flow assay, said method comprising:
(a) contacting a lateral flow test strip of the disclosure, or apparatus comprising same, with a biological sample;
(b) detecting the presence and/or level of the test analyte at the test portion;
(c) determining whether the lateral flow assay proceeded correctly on the basis of the presence or absence of control analyte(s) at the test portion(s); and
(d) on the basis of (c), determining whether the outcome at (b) is accurate.

As indicated herein, the presence or absence of control analyte(s) at the control portions(s) can be determined by determining the presence and/or level of detectable signal at the control portion(s). Likewise, the presence or absence of test analyte in the sample can be determined by determining the presence and/or level of detectable signal at the test portion. Thus, any discussion herein of the detection of the level and/or amount of control analyte(s) and/or test analyte is to be interpreted as encompassing the determination of the presence and/or level of the associated signal(s).

In accordance with a method of the disclosure in which the lateral flow test strip used comprises a single control portion i.e., a first control portion, as described herein, detection of a no signal at the first test portion following completion of the lateral flow assay may be indicative that the first control analyte, and thus the test sample, was present. On the other hand, detection of signal at the first test portion following completion of the lateral flow assay is indicative that the first control analyte, and thus the test sample, was not present.

In some example, such as for liquid samples, the test sample may be applied directly to the test strip. However, in other examples the test sample may be comprised in and mixed with a running buffer and the mixture applied to the test strip.

In accordance with a method of the disclosure in which the lateral flow test strip used comprises a first control portion and a second control portion as described herein, the following may apply
(v) detection of a signal at the second test portion and no signal or reduced signal at the first test portion (wherein reduced signal at the first test portion relative to signal at the second test portion) indicates that the lateral flow process proceeded correctly and the first control analyte was present in the sample applied to the test strip (“pass”);
(vi) detection of a signal at the first test portion and at the second test portion indicates that the lateral flow process proceeded correctly, but the first control analyte was not present in the sample applied to the test strip i.e., the test sample was absent (running buffer only was applied to the test strip) or the first control analyte in the test sample was degraded (“fail”);
(vii) detection of a signal at the first test portion and not at the second test portion indicates that the first control analyte was present in the sample applied to the test strip but the lateral flow process did not proceed correctly. Lack of signal at the second control portion indicates that the sample did not reach the second control portion and/or the second mobilisable labelled species or second immobilised capture reagent did not perform as intended (“fail”);
(viii) detection of no signal at the first or second test portions indicates that the lateral flow process did not proceed as intended. The lack of signal at the first and second control portions indicates that the first control analyte was not present in the sample applied to the test strip and/or the sample applied to the test strip did not reach the second control portion and/or the second mobilisable labelled species or second immobilised capture reagent did not perform as intended (“fail”).

It is envisaged that the apparatus and methods described herein may be modified to accommodate detection of any test analyte.

An apparatus in accordance with any aspect disclosed herein may comprise a single test strip or multiple test strips, as required. Where multiple test strips are present, features of the apparatus as disclosed herein may be present in each test strip or may be distributed across multiple test strips. Where multiple test strips are present, those test strips may be configured in series or in parallel. When in parallel, each test strip may be the same or may be different. For example, features of the apparatus as disclosed herein may be distributed across multiple test strips in parallel. The apparatus disclosed herein may comprise two or more test strips in parallel, wherein one of the test strips may be a test strip of the apparatus disclosed herein and the other one or more test strips may be configured to detect the target analyte using a competition assay e.g., as described in WO2005/059547.

The apparatus according to one or more aspects of the present disclosure may be provided in the form of a kit. In one example, a kit may comprise lateral flow test strip or an apparatus according to one or more aspects of the present disclosure and instructions for use. The test kit may further comprise a lateral flow assay running buffer.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1 shows a top view configuration of the test strip according to one embodiment of the present disclosure which comprises a single “active” control portion.

FIG. 2 shows a top view configuration of the test strip of FIG. 1 and schematic representations of pass and fail results when in use.

FIG. 3 shows a top view configuration of the test strip according to one embodiment of the present disclosure which comprises an first control portion (“active control”) and second control portion (“internal control”).

FIG. 4 shows a top view configuration of the test strip of FIG. 3 and schematic representations of pass and fail results when in use.

FIG. 5 shows an oblique view of a test device according to one embodiment of the present disclosure.

FIG. 6 shows a cross-sectional view of the test device of FIG. 5 along line A-A of FIG. 5.

FIG. 7 shows a schematic representation of a reading apparatus used in the test device of FIG. 6.

FIG. 8 is graphic representation of an HSA sandwich assay using gold nanoparticles as a detectable label.

FIG. 9 provides representative data showing the detection of HSA protein with gold labels in a lateral flow assay. As illustrated, a limit of detection of 5 ng/mL could be demonstrated in buffer (blue histogram). Positive control line (red histogram) demonstrates successful functionalization of the gold labels

FIG. 10 provides the results of a lateral flow assay using anti-HSA polyclonal antibody as a capture reagent at the control line to detect HSA as an active control. A hook effect was evident where HSA was present at concentrations higher than 100 μg/mL.

FIG. 11 provides the results of a lateral flow assay using anti-α-human IgG antibody as a capture reagent at the control line to detect human IgG as an active control. This illustrates how differential absorbance measurement provides a digital signal of the presence/absence of the test sample.

FIG. 12 illustrates the non-specific accumulation of Supernova particles at the test lines (top panel) and of gold nanoparticles (bottom panel). Fluorescence intensity and absorbance across the test strips was measured with a CAMAG scanner.

FIG. 13 illustrates the level of binding of HSA-coated gold nanoparticles, as determined by intensity fluorescence signal, at the C1 and C2 control lines in the (A) absence of test sample containing HSA, and (B) presence of test sample containing HSA. C1 is provided as a reference and has no capture reagent immobilised and C2 has anti-HSA antibodies immobilised.

FIG. 14 shows the dose-dependency profile of the relative change in signal at the C2 control line with increasing loading of mucus samples.

FIG. 15 illustrates that the active competitive control assay approach of the disclosure work well with other particle types compatible with lateral flow assays, such as 200 nm blue latex particles. Shown is a normalised response of 200 nm blue latex particles conjugated to either HSA or human IgG in a competitive assay format. Final concentration of analyte in dropper was calculated using normal range of analyte in human sera, assuming only 1 μL of sample was diluted in 400 μL of lysis buffer. Signal was measured using a CAMAG TLC Scanner 4 with an excitation wavelength of 660 nm.

FIG. 16 is a schematic illustrates the covalent coupling of proteins onto amine-functionalised blue latex particles via glutaraldehyde activation.

FIG. 17 illustrates the HFT signal responses from two lots (Lot A and lot B) of glutaraldehyde-activated 200 nm blue latex particles coupled to HSA. “HSA” samples contained 0.5% v/v human serum diluted in lysis buffer. “No HSA” samples contained lysis buffer only.

FIG. 18 is a schematic of one embodiment of the HFT test strip design with two test lines for influenza nucleoprotein (T1 and T2) and two control lines (C1 and C2), wherein the capture reagent at 1l is a mouse anti-HSA antibody and the capture reagent at C2 is a goat anti-chicken IgY antibody.

FIG. 19 shows representative profiles obtained using the HFT test strip of FIG. 19 with a blank sample (no human mucus) and a human nasal swab sample. Note: signals have been normalised to 100% for both C1 and C2 at time-point 51 (approx. 2 min post-conjugate wave detection).

FIG. 20 is a schematic illustrating the interpretation of results at the control lines (C1 and C2). As illustrated, a fluorescence profile which is indicative of a successful swab sample is signal detected at C2 only. Any other fluorescence profile is indicative of a test error.

FIG. 21 provides an exemplary dataset of volunteer human nasal swab samples (n=36) and buffer only samples (n=37)) from two different lots of co-coupled HSA+IgY latex particles.

### DETAILED DESCRIPTION

Lateral flow tests typically require validation by an internal control line. In traditional lateral flow (not accretion-based assays), unbound labels flowing downstream of the test lines are captured by an anti-species (e.g. anti-mouse) antibody at a control line. The appearance of a detectable signal at a control line provides evidence that the lateral flow test has run properly acting as positive reinforcement for the user in case of a negative test outcome, where otherwise no band would appear. The control also provides some indications that the biological components on the lateral flow test strip remained active during transport and storage. In certain instances, for example when a test is destined for home use, the test could benefit from a more active control. For example, instead of simply capturing unbound labels, an active control may specifically recognise a biomarker which is present in the biological sample. However, as discussed above, the inventors have recognised that the traditional internal or active controls are susceptible to the “hook effect” or “prozone effect” when a test analyte is present in intermediate to high concentrations, thereby leading a user to believe that the lateral flow test failed when in fact it did not.

The present disclosure provides lateral flow test strips and devices that include controls which are not susceptible to the “hook effect”. This is achieved, in part, by inclusion of an “active control” which relies on a competition assay to detect the presence or absence of a control analyte in a sample (or running buffer comprising same). This active control design may permit more accurate validation of test results, particularly when the test sample and/or the test analyte comprised therein is present in an abundant amount. The inventors have demonstrated the effectiveness of this approach using human serum albumin (HSA) as the active control analyte, since it is the most abundant protein in human mucus. The inventors found that the concentrations of HSA in a sample were so high as to make it an unsuitable control analyte for use in a lateral flow assay which relies on a sandwich assay format. This is because (as described herein) the control test line and the gold/latex particle surface are exposed to quantities of HSA so large that both surfaces are rapidly coated by the protein, thereby incapacitating the antibodies from forming a sandwich (i.e., “the hook effect”). As an alternative to the sandwich assay format, the inventors adopted a so-called competitive assay, where the labelled particles (e.g., gold or latex nanoparticles) bind directly to the sensor surface at the control line in absence of the target analyte. Whereas the presence of the target analyte triggers a competition that leads to a progressive decrease in signal or absence of signal at the control line. This approach was found to work well in the presence of high levels of HSA, thereby mitigating the “hook effect”.

One potential issue with an active control approach which relies on a competitive assay is the lack of positive feedback provided to the user (i.e., lack of detectable signal at the control line) when a negative test result genuinely occurs. The inventors therefore designed a lateral flow assay which combines an active control based on a control analyte present in the test sample e.g., HSA, with a further downstream internal control to help inform the user that (i) the test has been manufactured correctly, (ii) the detector particles are functional and (iii) the test has run to completion. Downstream internal controls of this type commonly rely on an anti-species capture antibody which directly binds detector particles conjugated with antibodies from the corresponding host species. For example, an anti-mouse capture antibody may be a suitable assay control in a lateral flow assay which uses mouse antibodies conjugated to their detector particles. However, the inventors have found that an anti-mouse capture antibody may not be a suitable assay control in all circumstances for two reasons: (i) the fluorescent detector particles commonly contain mouse antibodies which would compete with internal control particles, and (ii) mouse serum often is added to lateral flow tests as a blocking agent and this would rapidly saturate the anti-mouse capture line. For this reason, the inventors have incorporated a an internal control based on chicken IgY antibody as the control analyte. The inventors have discovered that chicken IgY has several advantages for the development of an internal control: (i) it is readily produced and extracted from chicken eggs in high yield, (ii) it is structurally different from mammalian IgG antibodies and has thus has no cross-reactivity with known human interferants such as complement, rheumatic factors or Fc-receptors, and (iii) several anti-species capture antibodies raised against chicken IgY are commercially available. Furthermore, the inventors have also found that in embodiments where both control analytes (e.g., HSA and chicken IgY) are co-coupled onto the same batch of gold or latex particles, every particle is capable of binding to either of the control lines.

### General Techniques and Definitions

Unless specifically defined otherwise, all technical and scientific terms used herein shall be taken to have the same meaning as commonly understood by one of ordinary skill in the art (e.g. in immunology, molecular biology, immunohistochemistry, biochemistry and pharmacology).

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

Lateral flow test strips and devices

The lateral flow test strip according to any one or more embodiments of the present disclosure may be formed of any material which permits flow of a liquid sample therethrough by capillary action and which is known to be suitable for use in lateral flow devices. Such materials have been widely used in commercially-available diagnostic tests e.g., influenza tests and pregnancy/conception tests, and will be known to a person skilled in the art. One such exemplary material may be a nitrocellulose membrane.

The lateral flow test strip may comprise a label holding portion and a first control portion. The one or more test strips may also comprise a sample receiving portion, a test portion and/or a second control portion. The size of each of the label-holding portion, the first control portion, the test portion, the sample receiving portion and the second control portion may be adapted as necessary. For example, the precise dimensions of each may be adapted according to the particular dimensions of the lateral flow test strip used and/or the dimensions of the apparatus with which the test strip may be used.

The label-holding portion and the first control portion may be configured on the lateral flow test strip such that, in use, a biological sample taken from a subject, or a LFA running buffer comprising same (collectively the “sample”), contacts the label-holding portion before the first control portion. The sample may contact the sample-receiving portion before the label-holding portion. The sample may contact the first control portion after contacting the test portion. In accordance with example in which the lateral flow test strip comprises a second control portion, the sample may contact the second control portion after contacting the first control portion. Alternatively, the sample may contact the second control portion before contacting the first control portion but after contacting the test portion. Alternative configurations are possible, including configurations where multiple strips are present.

As used herein, the terms “downstream” and “upstream”, when referring to the location of the various portions of the test strip, will be understood to mean relative to the direction of flow of the sample through or along the test strip.

The lateral flow test strip according to one or more embodiments of the present disclosure may also comprise a fluid sink, which may act to draw the sample through or along the one or more test strips.

As described herein, the lateral flow test strip of the disclosure may comprise one or more mobilisable labelled species and one or more immobilisable capture reagents configured to bind specifically to one of the mobilisable labelled species, either directly or indirectly e.g., via an attached or conjugated binding partner. The term “mobilisable” is used to indicate that the labelled species is capable of moving with the biological sample, or LEA running buffer comprising same, from the label-holding portion to the first and/or second control portion(s), as appropriate. The mobilisable labelled species may be deposited at the label-holding portion prior to use of the test strip by any suitable means known in the art. Conversely, the term “immobilised”, as used with respect to a capture reagent of the test strip of the disclosure, means the reagent is attached to the lateral flow test strip (e.g., at a control portion or test portion) such that lateral flow of fluids through or along the test strip during an assay process will not dislodge the reagent. The capture reagent may be immobilised by any suitable means known in the art.

As described herein, the lateral flow test strip may comprise a first mobilisable labelled species capable of binding to a first control analyte or which mimics at least one binding property of a first control analyte. The first mobilisable labelled species will also be capable of binding to the first immobilised capture reagent either directly or indirectly. The lateral flow test strip of the disclosure may further comprise a second mobilisable labelled species capable of binding to a second immobilised capture reagent. Alternatively, the lateral flow test strip of the disclosure may comprise a single mobilisable labelled species which is bound to both a first control analyte and a second control analyte. In each of the foregoing, the or each mobilisable labelled species may be located at or on the label-holding portion of the lateral flow test strip.

Examples of suitable mobilisable labelled species include, but at are not limited to, labelled antibodies, labelled proteins, latex beads or nanoparticles. In accordance with one example in which the first control analyte is HSA and the first mobilisable labelled species is capable of binding to the first control analyte, a suitable first mobilisable labelled species may be an anti-HSA antibody. The cognate first immobilised capture reagent at the first test portion may be HSA. In accordance with another example in which the first control analyte is HSA and the first mobilisable labelled species mimics at least one binding property of the first control analyte, a suitable first mobilisable labelled species may be HSA. The cognate first immobilised capture reagent at the first test portion may be an anti-HSA antibody. Although certain embodiments are described herein with reference to HSA as the first control analyte, a skilled person will appreciate that the first control analyte may be any analyte which is present, and preferably abundant, in a test sample. Examples of suitable types of analytes include, but are not limited to molecules, group of molecules or compounds of natural or synthetic origin (e.g., drugs, hormones, enzymes, growth factor antigens, antibodies, haptens, lectins, apoproteins, cofactors etc) which are capable of being bound and immobilised on the test strip using a suitable capture reagent. Where a second mobilisable labelled species is present on the test strip of the disclosure, the second mobilisable labelled species may be an analyte which is not typically present in a test sample (a second control analyte). The second mobilisable labelled species may be chicken IgY, for example. In accordance with this example, the second immobilised capture reagent may be an anti-species capture antibody raised against chicken IgY. However, a skilled person will appreciate that other immunoglobulins may be used in place of chicken IgY. Preferably the second control analyte is an immunoglobulin which is structurally different from mammalian IgG antibodies and has no cross-reactivity with known interferants e.g., in humans, such as complement, rheumatic factors or Fc receptors. The second control analyte may also be selected on the basis that anti-species capture antibodies raised against the second control analyte are commercially available. For example, in the case of chicken IgY, several anti-species capture antibodies raised against chicken IgY are commercially available e.g. goat anti-chicken IgY, donkey F(ab′)2 anti-chicken IgY, rabbit F(ab′)₂ anti-chicken IgY and monoclonal mouse anti-chicken IgY.

The disclosure also provides a lateral flow test strip comprising a mobilisable labelled species which is bound to both a first control analyte and a second control analyte. In accordance with this embodiment, the mobilisable labelled species may be, for example, a latex bead or nanoparticle conjugated to a first control analyte e.g., HSA, and a second control analyte e.g., chicken IgY. Exemplary first and second control analytes are described herein with reference to other embodiments and shall be taken to apply mutatis mutandis to this and any other embodiment or example of the disclosure unless specifically stated otherwise. In accordance with one example in which the first control analyte is HSA and the second control analyte is chicken IgY, the first mobilisable capture reagent may be an anti-HSA antibody and the second immobilised capture reagent may be an anti-species capture antibody raised against chicken IgY. However, the choice of cognate control analytes and cognate capture reagents may be varied as required.

In each of the foregoing examples, the capture reagent(s) immobilised at the control portion(s) may be any one of more agents having the capacity to bind to a mobilisable labelled species on the test strip, either directly or indirectly via a control analyte conjugated thereto, and thereby form a binding pair or complex. Some examples of such binding pairs, binding partners or complexes include, but are not limited to, an antibody and an antigen (wherein the antigen may be, for example, a peptide sequence or a protein sequence); complementary nucleotide or peptide sequences; polymeric acids and bases; dyes and protein binders; peptides and protein binders; enzymes and cofactors, and ligand and receptor molecules, wherein the term receptor refers to any compound or composition capable of recognising a particular molecule configuration, such as an epitopic or determinant site.

As used herein, the term “binding partner” refers to any molecule or composition capable of recognizing and binding to a specific structural aspect of another molecule or composition. Examples of such binding partners and corresponding molecule or composition include, but are not limited to, antigen/antibody, hapten/antibody, lectin/carbohydrate, apoprotein/cofactor and biotin/(strept)avidin.

In some examples, the lateral flow test strip of the disclosure also comprises one or more immobilised capture reagents configured to bind to a test analyte of interest in a sample. The one or more capture reagents configured to bind to a test analyte of interest may be immobilised at a test portion of the lateral flow test strip. The test analyte may be any analyte of interest in a sample. Suitable test analytes to be detected using a lateral flow test strip of the disclosure include, but are not limited to, antibodies to infectious agents (such as influenza, HIV, HTLV, Helicobacter pylori, hepatitis, measles, mumps, or rubella for example), antigens from infectious agents, cocaine, benzoylecgonine, benzodizazpine, tetrahydrocannabinol, nicotine, ethanol theophylline, phenytoin, acetaminophen, lithium, diazepam, nortryptyline, secobarbital, phenobarbital, methamphetamine, theophylline, testosterone, estradiol, estriol, 17-hydroxyprogesterone, progesterone, thyroxine, thyroid stimulating hormone, follicle stimulating hormone, luteinizing hormone, human chorionic gonadotropin hormone, transforming growth factor alpha, epidermal growth factor, insulin-like growth factor I and II, growth hormone release inhibiting factor, IGA and sex hormone binding globulin; and other analytes including antibiotics (e.g., penicillin), glucose, cholesterol, caffeine, cotinine, corticosteroid binding globulin, PSA, or DHEA binding glycoprotein.

It will be understood by those skilled in the art that the test strip of one or more embodiments of the present disclosure may be configured for use with a variety of different types of test samples. The choice of sample will in part be governed by the test analyte to be detected. A skilled person will understand that the sample should be chosen to be one in which it is suspected that the test analyte may be present. In addition, the choice of sample will be governed by the first control analyte which will act as an active control or vice versa. The sample may be a fluid sample. The test sample may be a biological sample. Biological samples which may be used in accordance with the lateral flow test strip of one or more embodiments of the present disclosure include, for example, blood, serum, plasma, urine, vaginal discharge and/or amniotic fluid and mucus. Medically relevant substances (e.g. analytes) can be found in blood (including antibodies, antigens, drugs, hormones, enzymes, metabolites, peptides and so forth), tears, sweat, and other secretions and exudate such as mucus. In one example, the test sample is a mucus sample. The test sample may also comprise, or be comprised in, a lateral flow assay (LFA) running buffer to aid flow of the sample through or along the test strip.

Of course, a person of ordinary skill in the diagnostic arts will appreciate that the lateral flow test strip of the disclosure may be configured for use in applications outside of human medicine, including, for example, veterinary, agricultural, agronomical and environmental applications. In accordance with these other areas of application, a person skilled in the art will be able to select appropriate control analyte(s) based on the test sample being relied upon, as well as appropriate capture reagents. For example, the lateral flow test strip of the disclosure may be configured to detect a test analyte in a test sample obtained from a plant, animal or environmental source. In accordance with an example in which the test sample is obtained from an animal, the test sample may be any of the biological samples described above with respect to human, such as a mucus sample, a blood sample or component part thereof, or a urine sample. In accordance with an example in which the test sample is plant based, the test sample may be a plant tissue e.g., leaf, seed, fruit or roots, or one or more components obtained from the plant tissue e.g., oil, protein, DNA, RNA or combinations thereof. In accordance with an example in which the test sample is an environmental sample, the sample may be a water sample or an eluate obtained from a soil sample.

A person skilled in the art will appreciate that the mobilisable species may be labelled by any suitable means known in the art. For example, the label may be conjugated directly to the mobilisable species, or the label may be conjugated to the mobilisable species via a linker. The attachment of the label can be through covalent bonds, adsorption processes, hydrophobic and/or electrostatic bonds, as in chelates and the like, or combinations of these bonds and interactions and/or may involve a linking group. In some examples, the mobilisable species is a detectable label to which the control analyte(s) is/are attached.

Any suitable detectable label known in the art may be used. Examples of suitable labels include, but are not limited to, particulate labels, radiolabels, fluorescent labels, enzymatic labels and imaging agents. For example, the labels may comprise latex or gold. The labels may be latex beads (of any colour, including of two or more distinguishable colours) or may be nanoparticles. Any suitable nanoparticle may be used. For example, the nanoparticle may be a magnetic particle, a selenium nanoparticle, a silver nanoparticle, a gold nanoparticle or a carbon nanoparticle. The labelled species may be a latex particle, a glutaraldehyde-activated latex particle or a nanoparticle aggregate. The fluorescent labels may comprise one or more quantum dots. Where the lateral flow test strip incorporates multiple fluorescent molecules, the respective molecules may be selected to fluoresce at different wavelengths e.g., upon excitation by light, to enable differential detection of two or more analytes in the sample. The labels may be reflective. Where the lateral flow test strip incorporates multiple reflective molecules, the respective molecules may be selected to reflect light at different wavelengths to enable differential detection of two or more analytes in the sample.

Any suitable immobilised capture reagents may be used at the control portion(s) and the test portion of the test strip. Capture reagents used in accordance with one or more embodiments of the present disclosure may be any one of more agents having the capacity to bind an analyte of interest, be that a control analyte or a test analyte, and thereby form a binding complex. Some examples of such binding pairs or complexes include, but are not limited to, an antibody and an antigen (wherein the antigen may be, for example, a peptide sequence or a protein sequence); complementary nucleotide or peptide sequences; polymeric acids and bases; dyes and protein binders; peptides and protein binders; enzymes and cofactors, and ligand and receptor molecules, wherein the term receptor refers to any compound or composition capable of recognising a particular molecule configuration, such as an epitopic or determinant site.

In accordance with an example in which a mobilisable labelled species is capable of binding to a control analyte e.g., the mobilisable labelled species is an antibody against the control analyte or is attached thereto, the cognate capture reagent which is immobilised to the test strip (i.e., the immobilised capture reagent) may be the respective control analyte or an analogue or derivative thereof which mimics at least one binding property of the control analyte. If, on the other hand, the mobilisable labelled species is the control analyte or an analogue or derivative thereof which mimics at least one binding property of the control analyte (or is attached thereto), the cognate capture reagent which is immobilised to the test strip (i.e., the immobilised capture reagent) may be a species which is capable of binding to the mobilisable labelled species or to the control analyte e.g., an antibody against the control analyte. Accordingly, suitable immobilised capture reagents may include, but are not limited to, a control analyte or an analogue thereof which mimics at least one binding property of the control analyte to be measured or an antibody against a control analyte. In the context of the test portion of the lateral flow test strip, an immobilised capture reagent will be configured to specifically bind to the test analyte e.g., an antibody against the test analyte.

As used herein, the term “specifically bind”, “bind specifically” or similar may refer to a capture reagent that does not bind significantly (e.g., above background binding levels) to any sample components other than the desired component or analyte. Accordingly, a capture reagent which “binds specifically to HSA”, for example, may not bind significantly or at all to any other analytes or components in a sample other than HSA, if HSA is in fact present.

The skilled artisan will be aware that an “antibody” is generally considered to be a protein that comprises a variable region made up of a plurality of immunoglobulin chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgA1 and IgA2) or subclass. The term “antibody” also encompasses humanized antibodies, human antibodies and chimeric antibodies. As used herein, the term “antibody” is also intended to include formats other than full-length, intact or whole antibody molecules, such as Fab, F(ab′)_{2,} and Fv which are capable of binding the epitopic determinant. These formats may be referred to as antibody “fragments”. In accordance with the present disclosure, it will be expected that these antibody formats retain some ability to selectively bind to the analyte, as required, examples of which include, but are not limited to, the following:

(1) Fab, the fragment which contains a monovalent binding fragment of an antibody molecule and which can be produced by digestion of whole antibody with the enzyme papain to yield an intact light chain and a portion of one heavy chain;

(2) Fab′, the fragment of an antibody molecule which can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains;

(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific (see, for example, WO 94/07921 and WO 98/44001);

and

(6) Single domain antibody, typically a variable heavy domain devoid of a light chain.

Accordingly, an antibody used as a capture reagent in accordance with the present disclosure may include separate heavy chains, light chains, Fab, Fab′, F(ab′)2, Fc, a variable light domain devoid of any heavy chain, a variable heavy domain devoid of a light chain and Fv. Such fragments can be produced by recombinant DNA techniques, or by enzymatic or chemical separation of intact immunoglobulins.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof. In some cases, the intact antibody may have one or more effector functions.

An antibody used as a capture reagent in accordance with the present disclosure may be a humanized antibody. The term “humanized antibody”, as used herein, refers to an antibody derived from a non-human antibody, typically murine, that retains or substantially retains the antigen-binding properties of the parent antibody but which is less immunogenic in humans.

The immobilised capture reagents of the first and second control portions may therefore be antibodies. For example, where the first control analyte is HSA, the immobilised capture reagent of the first control portion may be an antibody configured to bind an epitope specific to human serum albumin HSA. For example, where the second control analyte or second mobilisable species is chicken IgY, the immobilised capture reagent of the second control portion may be an antibody which binds an epitope or region on chicken IgY. The immobilised capture reagent of the second control portion may be, for example, an anti-chicken IgY antibody capable of binding chicken IgY.

Suitable antibodies for use in accordance with the present disclosure are commercially available or otherwise known in the art. Furthermore, methods for determining the binding specificity and affinity of antibodies are known in the art, such that a skilled person could readily identify binding reagent which are suitable for use in accordance with the present disclosure.

In some embodiments, the lateral flow test strip of the disclosure may be present in, or configured for use with, a device or apparatus (collectively referred to as a “device”). The device in accordance with the present disclosure may be a device that operates as a single unit. For example, the device may be provided in the form of a hand-held device. The device may be a single-use, disposable, device. Alternatively, the device may be partly or entirely re-usable. While in some embodiments the device may be implemented in a laboratory, the device may designed as a ‘point-of-care’ device, for home use or use in a clinic, etc. In other embodiments, the device may be implemented in the workplace e.g., for undertaking quality control or quarantine purposes. The device may provide a rapid-test device, with identification of target conditions being provided to the user relatively quickly, e.g., in under 10 minutes, 5 minutes or under 1 minute.

The device may comprise a single test strip, or multiple test strips. For example, a device comprising multiple test strips of the disclosure may comprise 2, 3, 4, 5, 6, 7, 8, 9, 10, 15, 20 or more test strips. The test strips may be arranged in parallel or in a series. The device may also be configured such as that test strips can be replaced after use.

A device in accordance with the present disclosure may also comprise a display, configured to present information about the results of the assay to a user.

The device in accordance with the present disclosure may comprise a reader to identify HSA at the first control portion and/or chicken IgY at the second control portion, for example. The reader may also be configured to identify a test analyte at the test portion. The reader may include one or more photodetectors capable of monitoring a light signal at the first and/or second control portions. The reader may also include one or more photodetectors capable of monitoring a light signal at the test portion.

As described herein, the signals at the first and/or second control portions, and the signal(s) at the test portion, which may be monitored or detected, may comprise light signals such as light reflection signals and/or fluorescent light signals or otherwise. The light signals may be generated as a result of detectable labels immobilised at the first and/or second control portions or the test portion reflecting light and/or fluorescing light. The device may comprise a light source that shines light on the first and/or second control portion to cause light reflection and/or fluorescing. Monitoring or detecting the presence and/or level of such light signals may comprise determining an absolute or relative intensity of the signals, for example. The absolute or relative intensity of the signals will be dependent on the number and type of detectable labels immobilised at the first and/or second control portions and the test portion. For example, in accordance with an embodiment described herein in which a test strip of the disclosure comprises a mobilisable labelled species bound to a first control analyte and a second control analyte, the detection of a low signal at the first control portion combined with a moderate or high signal at the second control portion may indicate a high level of the first control analyte e.g., HSA, in the sample. If, on the other hand, the signal at the first control portion is about equal to that at the second control portion, this may indicate an absence of test sample. It will be appreciated that the precise comparison of signals at the first and second control portions may depend on the particular affinities, quantities, and other properties of the immobilised capture reagents at the first and second control portions.

### Methods and use

The lateral flow test strip or device comprising same according to any one or more embodiments of the present disclosure may be used in a method of detecting an analyte in a test sample. More specifically, use of the lateral flow test strip or device in a method of detecting an analyte in a test sample may permit a determination to be made as to whether the test strips disclosed herein have worked correctly in the lateral flow assay, and whether a valid test result is obtained when performing the method. The methods may be carried out in a home environment, in a laboratory setting, in a clinical setting or other environment. The methods may comprise using a lateral flow test strip or device of an embodiment as disclosed herein.

In accordance with aspects in which a lateral flow test strip of the disclosure comprises a single control portion (i.e., a first control portion), the absence of, or a reduction in, detectable signal at the first control portion during use may be indicative that the lateral flow assay has been performed correctly. This is because, as the test sample flows through the test strip to the first test portion, the first control analyte comprised therein competitively binds to either the first mobilisable labelled species or the first immobilised capture reagent as appropriate (depending on which is configured to bind to the first control analyte), thereby preventing or reducing binding of the first mobilisable labelled species to the first immobilised capture reagent. A reduction in detectable signal at the first control portion may be determined relative to the level, or expected level, of detectable signal that would otherwise have been present at the first control portion in the absence of the first control analyte being present in the sample. Conversely, where the first control analyte is absent from the sample or unable to bind to the first immobilised capture reagent (e.g., in circumstances where only running buffer has travelled through the lateral flow test strip or the control analyte is degraded), the first mobilisable labelled species will be free to bind to the first immobilised capture reagent during the lateral flow process. This will result in a detectable signal at the first control portion.

In accordance with aspects in which a lateral flow test strip of the disclosure which further comprise a second mobilisable labelled species and a second control portion (i.e., dual controls), during use, the detection of signal at the second control portion may be indicative that the sample (optionally comprised in or comprising a running buffer) has travelled through the test strip during the lateral flow process, irrespective of whether the first control analyte is present. In this way, the second control portion (the internal control) provides positive feedback to the user in the event that no signal is detected at the first control portion (the active control). Accordingly, a lateral flow test strip of the disclosure with dual controls having first and second mobilisable species may be configured such that, in use:
(i) detection of a signal at the second control portion and no signal at the first control portion indicates that the lateral flow process proceeded correctly and the first control analyte was present in the sample (“control pass”);
(ii) detection of a signal at the first control portion and at the second control portion indicates that the lateral flow process proceeded correctly, but the first control analyte was not present in the sample (“control fail”);
(iii) detection of a signal at the first control portion and not at the second control portion indicates that the first control analyte was present in the sample but the lateral flow process did not proceed correctly e.g., the sample did not reach the second control portion and/or one or more of the test strip components did not perform correctly (“control fail”);
(iv) detection of no signal at the first or second control portions indicates that the lateral flow process did not proceed correctly e.g., the first control analyte was not present in the sample and/or the sample did not reach the second control portion and/or one or more of the test strip components did not perform correctly (“control fail”).

As described herein, another aspect of the disclosure provides a lateral flow test strip which comprises: a) a mobilisable labelled species which is bound to a first control analyte and a second control analyte; b) a first control portion comprising a first immobilised capture reagent, wherein the first immobilised capture reagent is configured to specifically bind to the first control analyte on the labelled species; c) and a second control portion comprising a second immobilised capture reagent, wherein the second immobilised capture reagent is configured to specifically bind to the second control analyte on the labelled species; wherein the first control analyte is an analyte which is typically present in a test sample e.g., HSA, and wherein the second control analyte is an analyte which is not typically present in the test sample e.g., IgY.

During use, and in circumstances where the first control analyte is present in the test sample, the mobilisable labelled species is less able or unable to bind to the first immobilised capture reagent at the first control portion because of competitive binding with the first control analyte in the sample. The mobilisable labelled species therefore continues to migrate through the test strip towards the second control portion, where it can bind to the second immobilised capture reagent at the second control portion. This results in a profile in which signal is detectable at the second control portion indicating that the lateral flow process proceeded correctly, and a reduced signal (relative to that at the second control portion) or no signal is detectable at the first control portion indicating that the first control analyte was present in the sample (“control pass”).

During use, and in circumstances where the first control analyte is absent from the test sample (or degraded), the mobilisable labelled species is able to bind to the first immobilised capture reagent at the first control portion, and to the second immobilised capture reagent at the second control portion, in relatively equal proportions. That is, there is no competitive binding at the first control portion. This results in a profile in which signal is detectable at both the first and second control portions in relatively equal amounts e.g., 1:1 ratio, indicating that the lateral flow process proceeded correctly, but the first control analyte was absent from the test sample or degraded (“control fail”).

If, in either of circumstances above, the test sample did not migrate through the lateral flow test strip correctly to reach the second control portion and/or if one or more of the test strip components did not perform correctly e.g., if the mobilisable labelled species and capture reagent at the second control portion were unable to bind, then no signal would be detectable at the second control portion indicating that the lateral flow process did not proceed correctly (“control fail”).

Based on the result obtained during use of the lateral flow test strips as described herein, a determination may be made regarding whether the test strips have worked correctly in the lateral flow assay, and whether a valid test result is obtained when performing a diagnostic method on a test sample to detect a test analyte.

### Kits

The lateral flow test strip or device in accordance with the present disclosure may be provided in the form of a kit. Such kits may include one or more test strips or devices (which may be for the same or different analytes), and instructions for use. The instructions for use may provide directions on how to apply sample to the test strip or the device, the amount of time necessary or advisable to wait for results to develop, and details on how to read and interpret the results of the test. Such instructions may also include standards, such as standard tables, graphs or pictures for comparison of the results of a test. These standards may optionally include the information necessary to quantify analyte using the test device, such as a standard curve relating intensity of signal or number of signal lines to an amount of analyte therefore present in the sample. Alternatively, or in addition, a kit may comprise an device of one or more embodiments of the present disclosure and one or more test strips compatible for use in the device. In this respect, the device may be configured to allow removal of a used test strip from the casing after use and subsequent placement with a new test strip into the casing.

### DESCRIPTION OF EXEMPLARY EMBODIMENTS

### Exemplary embodiment 1

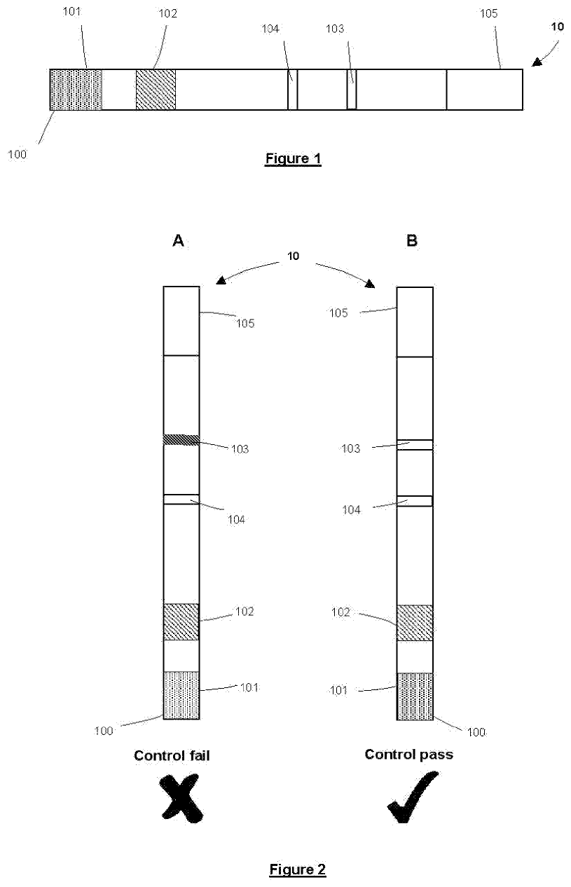

A lateral flow test strip according to one embodiment of the present disclosure is illustrated in FIG. 1 (test strip 10). The test strip 10 is a lateral flow test strip constructed of chemically-treated nitrocellulose, located on a waterproof substrate which is configured to (i) detect the presence of HSA in a test sample as an active control using a competitive binding assay, and (ii) detect the presence and/or amount of a test analyte of interest in the test sample. The test sample may be any human biological sample which typically contains HSA e.g., human mucus or blood or a component thereof. The biological sample may be added to a LFA running buffer (the biological sample and/or LFA running buffer collectively referred to as the “sample”) to aid its migration through or along the test strip 10.

Referring to FIGS. 1 and 2, the test strip 10 is a lateral flow test strip including different zones arranged sequentially along the length of the strip, including a sample receiving zone 101 at the sampling end 100, a label-holding zone 102, a control zone 103, test zone 104, and a sink 105. The zones 101-105 comprise chemically-treated nitrocellulose, located on a waterproof substrate 106. The arrangement of the zones 101-105 and substrate 106 is such that, when contacted with the sample receiving zone 101, the liquid sample is absorbed into the sampling receiving zone 101 and at least part of the sample travels under capillary action sequentially through the sample receiving zone 101, the label-holding zone 102, the test zone 104, the control zone 103 and accumulates finally at the sink 105.

this embodiment, the label-holding zone 102 comprises a label-conjugated control antibody (i.e., the mobilisable labelled species). The label-conjugated control antibody is an anti-HSA antibody designed to bind specifically to HSA, if present, in the sample or to HSA immobilised at the control zone 103 in the event that the antibody is not already bound to HSA from the sample. Accordingly, as the sample travels through the label-holding zone 102, HSA (if present in the sample) binds to the label-conjugated anti-HSA antibody to form a labelled control binding complex. If, on the other hand, HSA is not present in the sample, the label-conjugated anti-HSA antibody travels unbound through the test strip 10. The sample continues to travel along the test strip 10, through the test zone 104, the control zone 103, and ultimately migrating to the sink 105. If HSA is present in the sample, the formation of a labelled control binding complex will prevent the label-conjugated anti-HSA antibody from binding to the HSA immobilised at the control zone 103. If, on the other hand, the sample does not contain HSA or if the HSA is degraded in the sample, the sample containing the mobilised label-conjugated anti-HSA antibody will travel through the test strip 10 and, in the absence of being bound to HSA in the sample, will bind to the immobilised capture reagent (i.e., HSA) at the control zone 103.

Although this embodiment includes label-conjugated anti-HSA antibody as the mobilisable labelled species at the label-holding zone 102 and HSA as the immobilised capture reagent at the control zone 103, the competitive active control would also work if label-conjugated HSA was used as the mobilisable labelled species at the label-holding zone 102 and anti-HSA antibody was used as the immobilised capture reagent at the control zone 103.

In order to detect a test analyte of interest, the label-holding zone 102 may also comprise a mobilisable label-conjugated antibody designed to bind specifically to a test analyte of interest e.g., an influenza nucleoprotein (flu NP), if present in the sample to form a complex (hereinafter “labelled flu NP complex”). Accordingly, as the sample travels through the label-holding zone 102, flu NP present therein binds to the anti-flu NP antibody to form a labelled flu NP complex. The sample containing the labelled flu NP complex continues to travel though the test strip to the test zone 104 that contains immobilized compounds e.g., an antibody, capable of binding flu NP with high specificity and affinity. On contact, the immobilized compounds in the test zone 104 binds to the flu NP in the labelled flu NP complex to form a labelled flu NP sandwich. The sample continues through the test strip 10 to contact the control zone 103 as described above.

In this embodiment, the label-conjugated antibodies are labelled with different types of fluorescent quantum dots (QDs), configured to fluoresce at a different specific emission peak wavelengths following UV light excitation (e.g., first and second wavelengths of 525 and 800 nm, respectively). Of course, in alternative embodiments, other types of labels may be used in place of quantum dots, such as latex beads or gold particles, etc., and/or other specific emission peak wavelengths may be used.

As schematically illustrated in FIG. 2A, detectable signal at the control portion 103 is indicative of the presence of label-conjugated anti-HSA antibody bound to HSA at the control portion 103, and indicative that the sample being tested does not contain HSA. By contrast, a lack of detectable signal at the control portion 103 is indicative of the absence of label-conjugated anti-HSA antibody bound to HSA at the control portion 103 i.e., because the label-conjugated anti-HSA antibody was competitively bound by HSA in the sample, and therefore indicative that the sample being tested does contains HSA (FIG. 2B).

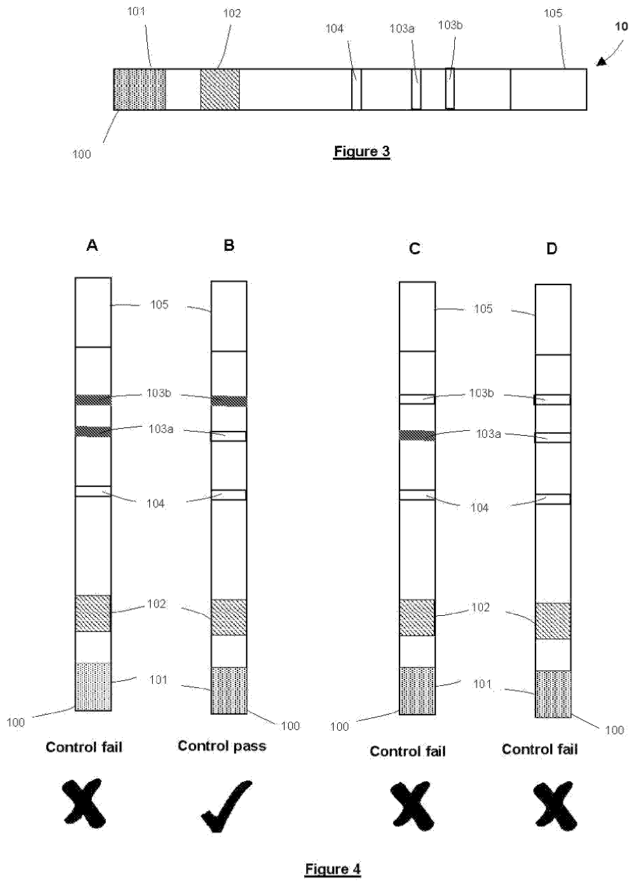

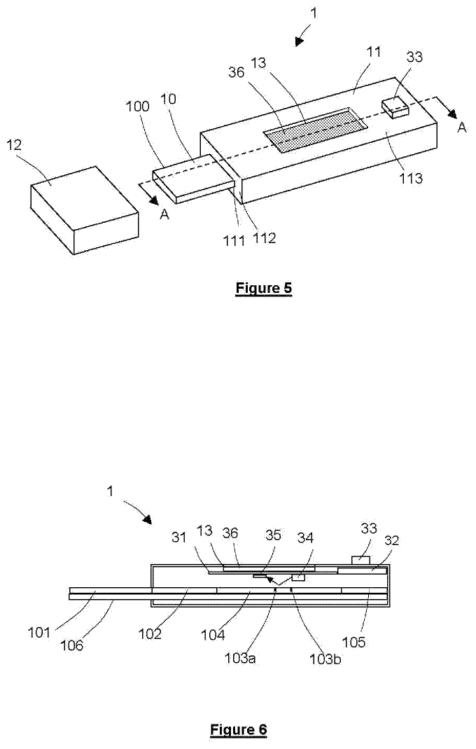

In addition to the “active control” at the control portion 103 configured to detect HSA in a test sample using a competitive binding assay, certain embodiments of the lateral flow test strip of the disclosure may further comprise a downstream “internal control” at the test zone to help inform the user that (i) the test strip has been manufactured correctly, (ii) the detector particles are functional and (iii) the FLA test has run to completion. Referring to FIGS. 3 and 4, the test strip 10 is a lateral flow test strip including different zones arranged sequentially along the length of the strip consistent with the embodiment described with reference to FIGS. 1 and 2, with the exception that the control zone 103 comprises a first control portion 103a (the “active control”)_and a second control portion 103b (the “internal control”).

In this embodiment, the label-holding zone 102 comprises a first label-conjugated control antibody (i.e., the first mobilisable labelled species) and a second label-conjugated control antibody (i.e., the second mobilisable labelled species) . The first label-conjugated antibody is an anti-HSA antibody designed to bind specifically to HSA, if present, in the sample or to HSA immobilised at the first control portion 103a in the event that the antibody is not already bound to HSA from the sample. The second label-conjugated antibody is a chicken IgY antibody designed to bind specifically to anti-chicken IgY antibody immobilised at the second control portion 103b. Accordingly, as the sample travels through the label-holding zone 102, HSA (if present in the sample) binds to the label-conjugated anti-HSA antibody to form a labelled control binding complex, which is carried with the label-conjugated chicken IgY antibody through the test strip 10. If, on the other hand, HSA is not present in the sample, the label-conjugated anti-HSA antibody travels unbound through the test strip 10 with the label-conjugated chicken IgY antibody. The sample continues to travel along the test strip 10, through the test zone 104, the control zone 103, and ultimately migrating to the sink 105. If HSA is present in the sample, the formation of a labelled control binding complex will prevent the label-conjugated anti-HSA antibody from binding to the HSA immobilised at the first control portion 103a. If, on the other hand, the sample does not contain HSA or if the HSA is degraded in the sample, the sample containing the mobilised label-conjugated anti-HSA antibody will travel through the test strip 10 and, in the absence of being bound to HSA in the sample, will bind to the immobilised capture reagent (i.e., HSA) at the first control portion 103a. Furthermore, provided that the sample is able to migrate all the way to the sink 105 (i.e., flow to completion) and provided that all of the components of the test strip 10 are functional, the label-conjugated chicken IgY antibody will bind to the immobilised capture reagent (i.e., anti-chicken IgY antibody) at the second control portion 103b. However, if the sample did not migrate as far as the second control portion 103b or if any of the internal control components e.g., mobilisable labelled species or immobilised capture reagent, are not functional, then the label-conjugated chicken IgY antibody will not bind to the immobilised capture reagent (i.e., anti-chicken IgY antibody) at the second control portion 103b.

As per the previous embodiment, the label-holding zone 102 may also comprise a mobilisable label-conjugated antibody designed to bind specifically to a test analyte of interest e.g., an influenza nucleoprotein (flu NP), if present in the sample to form a complex (hereinafter “labelled flu NP complex”). Accordingly, as the sample travels through the label-holding zone 102, flu NP present therein binds to the anti-flu NP antibody to form a labelled flu NP complex. The sample containing the labelled flu NP complex continues to travel though the test strip to the test zone 104 that contains immobilized compounds e.g., an antibody, capable of binding flu NP with high specificity and affinity. On contact, the immobilized compounds in the test zone 104 binds to the flu NP in the labelled flu NP complex to form a labelled flu NP sandwich. The sample continues through the test strip 10 to contact the control zone 103 as described above.

In this embodiment, the label-conjugated antibodies are labelled with different types of fluorescent quantum dots (QDs), configured to fluoresce at a different specific emission peak wavelengths following UV light excitation (e.g., first and second wavelengths of 525, 625 and 800 nm, respectively). Of course, in alternative embodiments, other types of labels may be used in place of quantum dots, such as latex beads, magnetic particles or gold particles, etc., and/or other specific emission peak wavelengths may be used.

As schematically illustrated in FIG. 4A, detectable signal at the first control portion 103a is indicative of the presence of label-conjugated anti-HSA antibody bound to the immobilised HSA at the first control portion 103a, indicating that the sample being tested does not contain HSA (e.g., because the sample is not present in the LFA running buffer or is degraded). Further, detectable signal at the second control portion 103b is indicative of the presence of label-conjugated chicken IgY antibody bound to the immobilised anti-chicken IgY antibody at the second control portion 103b, which indicates that the lateral flow assay proceeded correctly. Collectively, this control profile is indicative of a “fail” because of the lack of control analyte HSA in the sample.

As schematically illustrated in FIG. 4B, a lack of detectable signal at the first control portion 103a is indicative of the absence of label-conjugated anti-HSA antibody bound to the immobilised HSA at the first control portion 103a i.e., because the label-conjugated anti-HSA antibody was competitively bound by free HSA present in the sample. This is indicative that the sample being tested contains HSA. Further, detectable signal at the second control portion 103b is indicative of the presence of label-conjugated chicken IgY antibody bound to the immobilised anti-chicken IgY antibody at the second control portion 103b, which indicates that the lateral flow assay proceeded correctly. Collectively, this control profile is indicative of a control “pass” because the control analyte HSA was detected in the sample and the LFA proceeded to completion correctly.

As schematically illustrated in FIG. 4C, detectable signal at the first control portion 103a is indicative of the presence of label-conjugated anti-HSA antibody bound to the immobilised HSA at the first control portion 103a, indicating that the sample being tested does not contain HSA (e.g., because the sample is not present in the LFA running buffer or is degraded). A lack of detectable signal at the second control portion 103b is indicative that the label-conjugated chicken IgY antibody did not bind to the immobilised anti-chicken IgY antibody at the second control portion 103b, which indicates that the lateral flow assay did not correctly and/or to completion. Collectively, this control profile is indicative of a control “fail” because the LFA did not proceed to completion correctly.

As schematically illustrated in FIG. 4D, a lack of detectable signal at the first control portion 103a is indicative of the absence of label-conjugated anti-HSA antibody bound to the immobilised HSA at the first control portion 103a i.e., because the label-conjugated anti-HSA antibody was competitively bound by free HSA present in the sample. This is indicative that the sample being tested contains HSA. A lack of detectable signal at the second control portion 103b is indicative that the label-conjugated chicken IgY antibody did not bind to the immobilised anti-chicken IgY antibody at the second control portion 103b, which indicates that the lateral flow assay did not correctly and/or to completion. Collectively, this control profile is indicative of a control “fail” because the LFA did not proceed to completion correctly. In an alternative embodiment describe with reference to FIGS. 3 and 4, a first control analyte (i.e., HSA) and a first control analyte (i.e., chicken IgY) are co-coupled to the same mobilisable labelled species (i.e., nanoparticles) located at the label-holding portion 102 of the test strip 10. In accordance with this embodiment, every nanoparticle is capable of binding to either of the first and second control portions 103a, 103b. The configuration of the remaining components of the test strip 10 is the same as previously described for FIGS. 3 and 4.

In this embodiment, the label-holding zone 102 comprises a single mobilisable labelled species conjugated to a first control analyte (i.e., HSA) and a first control analyte (i.e., chicken IgY). The first control portion 103a has an anti-HSA antibody immobilised thereto and the second control portion 103b has an anti-chicken IgY antibody immobilised thereto. In this way, the mobilisable labelled species is able to bind to both the first and second control portions 103a, 103b. As the sample travels through the test strip, HSA (if present in the sample) binds to the anti-HSA antibody immobilised at the first control portion 103a thereby preventing or reducing binding of the mobilisable labelled species thereto. The remaining mobilisable labelled species is carried through the test zone 103 where the chicken IgY antibody binds to the anti-chicken IgY antibody immobilised at the second control portion 103b. This results in a control profile in which detectable signal is emitted from the second control portion 103b and, if detectable signal is emitted from the first control portion 103a at all, it is emitted at a reduced level than that emitted from the second control portion 103b. As illustrated in FIG. 4, this control profile indicates that the biological sample containing the control analyte is present and that the lateral flow process proceeded to completion correctly (control “pass”). If, on the other hand, HSA is not present in the sample, the mobilisable labelled species travels unbound through the test strip 10 with both the HSA and chicken IgY antibody coupled thereto available for binding to the immobilised capture reagents at first and second control portions, 103a 103b respectively. As the sample reaches the control zone 103, the mobilisable labelled species binds to the immobilised capture reagents at first and second control portions, 103a 103b in approximately equal proportions. As illustrated in FIG. 4, this control profile indicates that the biological sample containing the control analyte was not present (i.e., in the FLA running buffer) but that the lateral flow process proceeded to completion correctly (control “fail”). If, in either of the above scenarios, there is a lack of detectable signal at the second control portion 103b (as illustrated in FIG. 4C and 4D), this indicates that the label-conjugated chicken IgY antibody did not bind to the immobilised anti-chicken IgY antibody at the second control portion 103b, which indicates that the lateral flow assay did not proceed correctly and/or to completion (control “fail”).

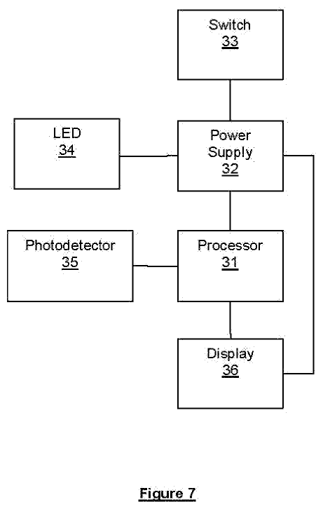

In an alternative embodiment, a test strip 10 of the disclosure may be used in combination with a device e.g., a handheld device, to assist in detection of a test analyte in a sample. An device according to an embodiment of the present disclosure is illustrated in FIGS. 5 and 6 (test device 1). The test device 1 is a hand-held device configured for use with a test strip 10 as illustrated in FIGS. 1-4 to (i) detect the presence or absence of a test analyte in a sample following performance of a LFA sandwich assay, and (ii) to validate the test result using controls of the test strip described herein.

The test device 1 includes an elongate lateral flow test strip 10 and a casing 11. The test strip 10 is partially housed in the casing 11 with a sampling end 100 of the test strip 10 protruding from an opening 111 in an end surface 112 of the casing 11, allowing sample to be received directly thereon. The sampling end 100 of the test strip 10 is coverable by a cap 12. The test device 1 also includes an LCD display 36 visible through an opening 13 in a top surface 113 of the casing 11 for displaying results of testing.

Referring to FIG. 7, a reading apparatus of test device 1 of the present embodiment is now described in more detail. The reading apparatus includes a printed circuit board having a processor 31, a power supply (battery) 32, a switch 33, a UV LED 34, a multi-wavelength photodetector 35 and the display 36. The LED 34 is configured to emit light in the UV spectrum (at about 300 to 400 nm) that is incident on the control portions 103a and 103b, and test portion 104, to cause excitation of any quantum dot labels located thereon. The multi-wavelength photodetector 35 in combination with the processor 31 is configured to detect the different intensities of light emitted from the quantum dots at different distinct wavelengths (if desired).

In use, the cap 12 is removed from sampling end 100 of the test strip and a liquid sample is directed onto the sample receiving zone 101. The cap 12 can be replaced and, after approximately 1 or 2 minutes, giving sufficient time for the lateral flow process to take place, the switch 33 can be depressed, causing flow of electricity from the power supply 32 to the LED 34, resulting in emission of UV light from the LED 34 that is incident on the control portions 103a, 103b and test portion 104 of the test strip 10. The UV light results in excitation of any or all of the quantum dots that may be immobilized as part of the labelled complexes at the control portions 103a, 103b and test portion 104 causing light emission at respective wavelength peaks. In combination with the multi-wavelength photodetector 35, the processor 31 is configured to determine the size of the emission peaks and identify from this (a) if the sample mix has arrived at the control portions 103a, 103b and labelling has been effective, and if yes, identify (b) the presence and optionally, an amount, of labelled test analyte present in the sample based on the intensity of light emission detected at portion 104.

While a manual switch 33 is described above, in alternative embodiments, switching may be automated. For example, switching may be configured to occur upon replacement of the cap 12 onto the casing 11 or due to fluid activation, as the sample travels through a fluid-activated switch that may be provided in the device.

The LED may be carefully calibrated to ensure that the light emission from the LED is consistent from one device to the next, ensuring that a degree of excitation of the quantum dots is consistent. Additionally, or alternatively, a calibration mechanism may be integrated into the device. A known quantity of quantum dots, configured to fluoresce at yet another wavelength, may be immobilized on the test strip, e.g. at a further test stripe. Depending on the intensity of the fluorescence detected from the known quantity of quantum dots, the processor may adjust its interpretation of the light emission from quantum dots on the labelled complexes. Additionally, or alternatively, multiple LEDs may be used to excite the quantum dots with a view to suppressing the overall effect of any rogue LEDs.

If, during use, it is identified there is insufficient amount of sample to reach the control zone, or if a “failed” control profile as illustrated in FIGS. 2 and 4 and described above is identified, the processor 31 is configured to cause the display 36 to present the words INVALID TEST. In this respect, the processor 31, in combination with the multi-wavelength photodetector 35, is configured to determine the size of the emission peaks at the control zone 103 and identify from this (i) if the sample has arrived at the control zone 103, and/or (ii) if labelling has been effective, and/or (iii) if biological sample is present and undegraded.

If, during use, it is identified there is sufficient amount of sample and labelling is effective, the processor 31 is configured to provide a determination that the sample contains the test analyte or not.

Since the device of the present embodiment is a hand-held device, the device may be used in the laboratory, the clinic, at home or in the workplace.

The device is configured to allow removal of a used test strip from the casing 10, via the opening 111, and allow placement of a new test strip into the casing 10, via the same opening 111. In alternative embodiments, the device may be entirely a single-use device.

### EXAMPLES

### Example 1—Development of an Improved Active Control for Lateral Flow Test Strips

### HSA-Based Active Control

Lateral flow tests typically require validation by an internal control line. In traditional lateral flow (i.e. not accretion), unbound labels flowing downstream of the test lines are captured by an anti-species (e.g. anti-mouse) antibody. The appearance of a control line provides evidence that the test has run properly acting as positive reinforcement for the user in case of a negative test outcome, where otherwise no band would appear. It also provides some indications that the biological components on the test trip remained active during transport and storage. In certain instances, for example when a test is destined for home use, the test could use a more informative active control. Instead of simply capturing unbound labels, an active control specifically recognises a biomarker present in the biological sample.

The Home Flu Test (HFT), also destined for home use and OTC sales, implements an active control. The most abundant proteins and candidate target markers for the HFT control line are human serum albumin (HSA) and immunoglobulins (IgG, IgA). IgA has been discarded after initial evaluation because of a non-negligible fraction of the population being IgA deficient.

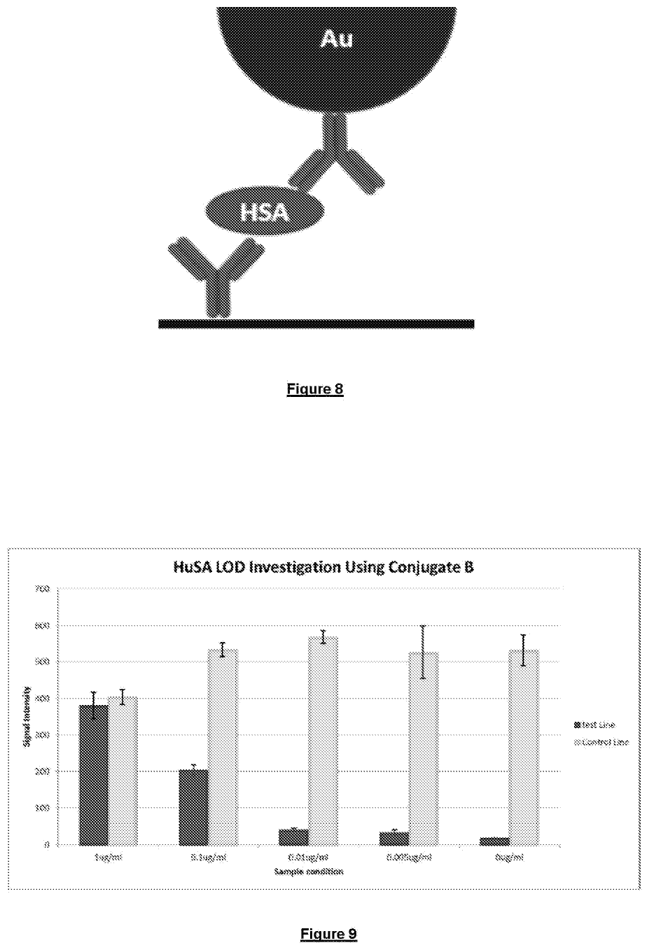

HSA is the most abundant protein in human mucus and was therefore evaluated on the HFT. Multiple antibodies were screened in a sandwich assay format. The anti-HSA antibodies were immobilised on the nitrocellulose strip and onto gold nanoparticles. In presence of mucus sample, both antibodies recognised the HSA protein, thus forming a functional sandwich (FIG. 8). The assay format is identical to the flu assay, where the nucleoprotein is trapped between two antibodies. With this format, spiked HSA in buffer could be detected with limit of detection of 5 ng/mL (FIG. 9). Successful conjugation of the anti-HSA antibodies to the gold nanoparticles was verified by including an anti-species control line to the test.

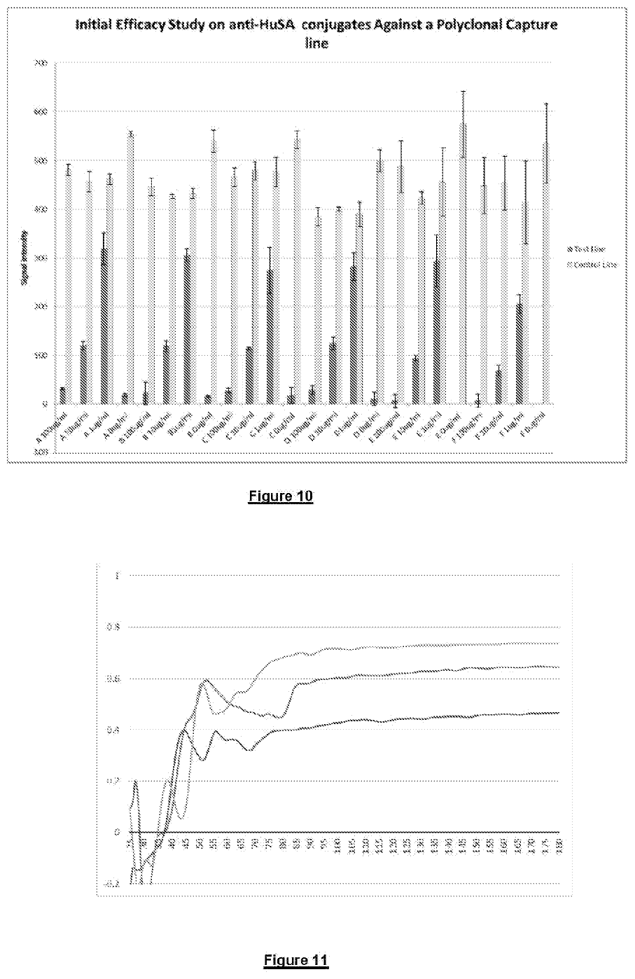

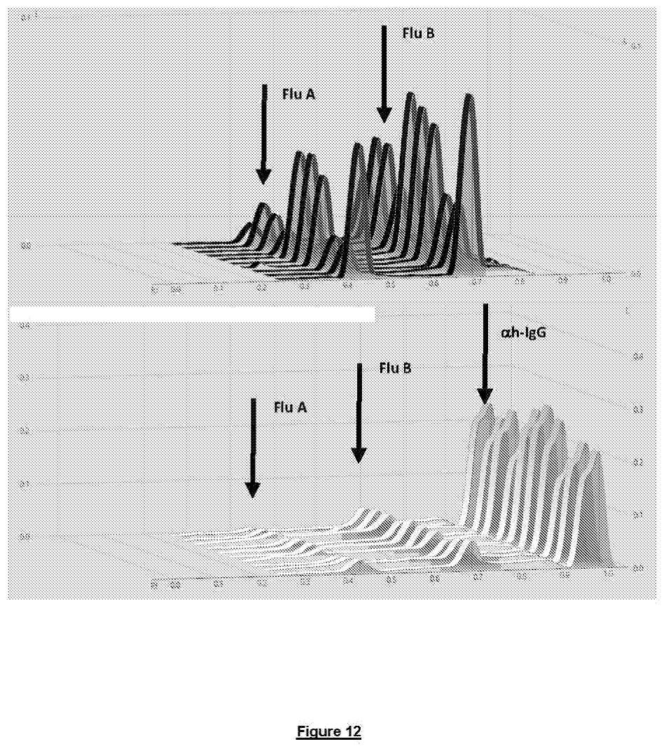

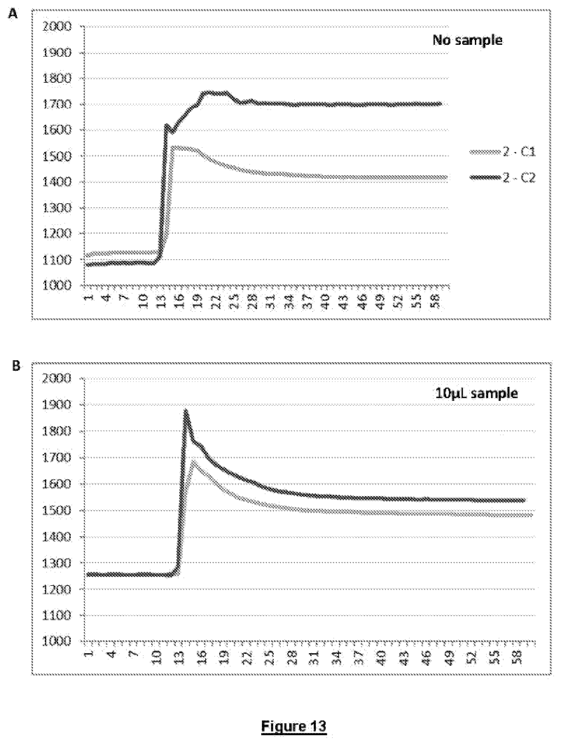

The results from this initial experiment indicated that the dynamic range was not suitable for a test control assay: reported values of HSA protein in nasal mucus is in the range of several mg/mL and well above saturation of the assay (see FIG. 10). The signal does in fact reach maximum values at 1 μg/mL and progressively decreases at higher concentrations (due to a “Hook effect”). The test lines and the particle surface are exposed to quantities of HSA so large that both surfaces are rapidly coated by the protein, thus incapacitating the antibodies from forming a sandwich (FIG. 10).

Based on this finding, a different target was selected for evaluation and possible implementation on the HFT: a-human IgG antibodies were immobilised on the C2 control line of the test strip. A combination of Supernova particles (i.e., nanoparticle aggregates) and anti-human IgG gold nanoparticles was deposited simultaneously on the conjugate release pad. In absence of the sample, the gold should flow past the control line without binding. If a mucus sample has been successfully applied, the gold particles will sequester immunoglobulins from the sample and accumulate at the control line. A differential absorbance measurement (that is, a measurement of the light absorbed by the gold particles compared with a section of bio-inactive nitrocellulose as reference) provided a digital signal of the presence/absence of the sample (FIG. 11).

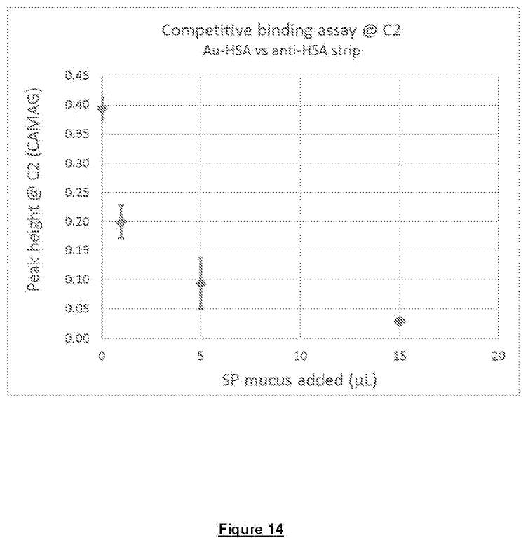

While this assay format was shown to be well within the range of IgGs found in human mucus, occasional but significant interference with Supernovas in mucus samples was observed (FIG. 12).

Fluorescence intensity and the absorbance across the test strips was measured with a CAMAG TLC scanner. Distinct peaks, due to non-specific binding of Supernova particles, could be observed at both flu test lines (FIG. 12). An absorbance scan of the same strips revealed gold nanoparticles absorbed non-specifically at the flu test lines. It is believed that the anti-human antibodies on the gold nanoparticles interacted with a subpopulation of immunoglobulins with affinity for the anti-nucleoprotein IgGs immobilised at the flu test lines. The variability in background response deriving from these non-specific interactions would severely affect the sensitivity of the HFT assay.

It was therefore decided to re-explore the use of HSA with a revisited assay format. It is well-known in assay development that a sandwich format as described in FIG. 8 can deliver very good sensitivities. For this reason, sandwich assays are the assay format of choice for many lateral flow based diagnostics. However, another approach is to develop a so-called competitive assay, where the labelled particles bind directly to the sensor surface in absence of the target analyte. Presence of the target analyte therefore triggers a competition that leads to a progressive decrease in signal or absence of signal.

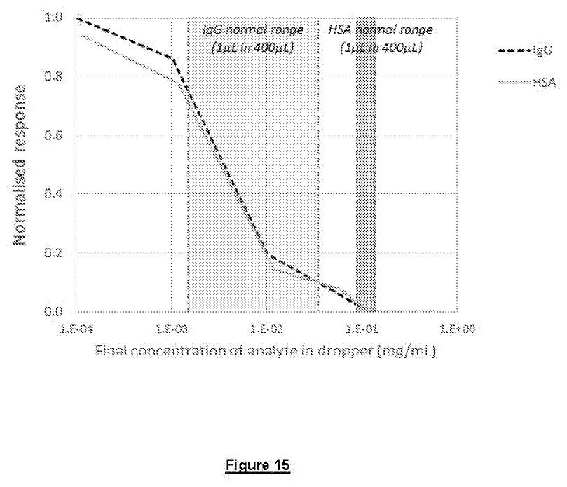

In the development of a competitive control assay format, we immobilized anti-HSA antibodies on the nitrocellulose and introduced HSA-coated gold nanoparticles in the assay system. The relative change in absorbance through the dry/wet transition was then observed when the sample reached the test strip and subsequent signal generation while the particles flowed through the strip. The signal at the C1 control line (blue line in FIG. 13A), where no capture reagents are immobilized, showed a signal increase in correspondence of the wet/dry transition and the passage of the conjugate wave. It is noteworthy that the negative signal has a monotonous decay until it reaches background value. Conversely, in absence of sample the gold particles accumulated on the C2 control line providing a stable response within 5 minutes (30 reads) from sample loading (FIG. 13A). The signal profile therefore increased from the wet/dry transition to the final equilibrium level, similar to the signal profile observed in presence of sample with the ah-IgG control line (FIG. 12).

Conversely, in presence of sample both C1 and C2 exhibited a similar signal profile that can be described by a monotonic wave form constantly decreasing towards the background level. The levels of HSA in human mucus were so elevated that the C2 test line was completely passivated and no binding of gold particles was observed.

The dose-dependency profile of the relative change in signal at the C2 control line with increasing loading of mucus samples is provided in FIG. 14.

Surprisingly, the morphology of the signal profile was significantly different when no sample was applied and could be discriminated even in absence of the reference signal at C1. Based on this observation, it was hypothesized that this sensing mechanism could be transitioned from differential to an absolute measurement, thus eliminating one LED at the C1 test line and therefore simplifying the device design.

The robustness of the assay was been verified for small sample volumes. Mucus volumes larger than 5 μL resulted in non-detectable gold signal at C2. At lower volumes the signal then rapidly converged to the “background” signal. The assay has also been validated on mucus samples from 4 different donors confirming that the sensing mechanism is robust and reproducible.

### Blue Latex Particles

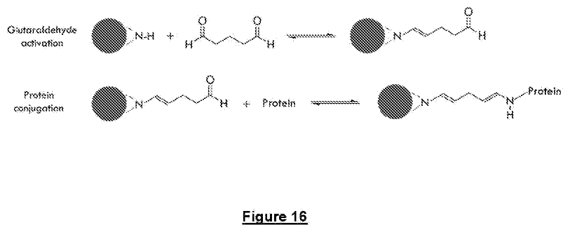

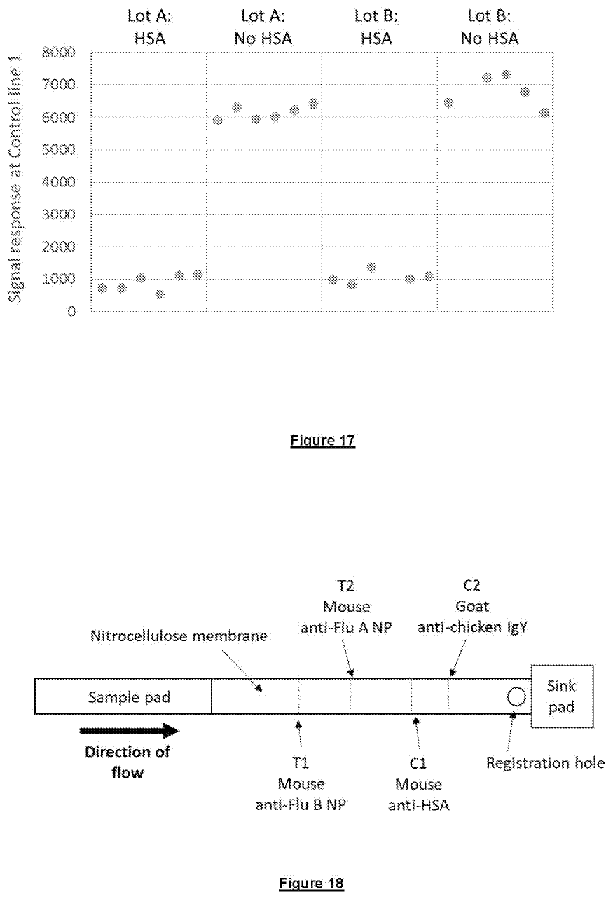

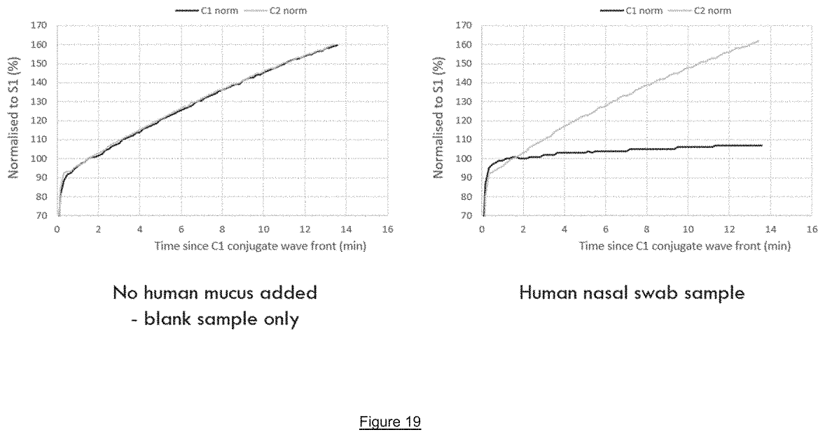

This competitive assay approach may be extended to further improve the utility of the control assay, particularly in a home-use environment. Some of these are described below.
1) It has been shown that this active competitive assay control approach is not limited to colloidal gold and can work very well with other particle types compatible with lateral flow assays, e.g., 200 nm blue latex particles (see FIG. 15). Absorbance due to colloidal gold can be measured using a green LED (λabs: 530 nm) while absorbance due to blue latex particles can be measured using a red LED (λabs: 630 nm). Either option can be incorporated into the Ellume Home Flu Test.
2) In typical lateral flow tests, these detector particles would be dried onto a conjugate release pad and assembled within the test strip itself. The inventors have developed an alternative format, termed the accretion method, whereby the detector particles are placed on a release pad in the sample dropper. In this format, the particles are eluted when: (i) the processing solution (lysis buffer) is added into the dropper, and (ii) the nozzle containing the human swab sample is screwed onto the dropper. This produces a homogeneous solution of detector particles mixed with sample which improves the consistency and sensitivity of the assay. This also has the added benefit of removing the conjugate release pad and thus simplifying the fluidics of the overall test strip.
3) The inventors have demonstrated several immobilisation approaches for physisorption or covalent attachment of HSA to 200 nm blue latex particles with a variety of surface functional groups, e.g. carboxyl, amine or bare polystyrene. The preferred approach is based on the method of Wood and Gadow (1983) J Clin Chem Clin Biochem, 21: 789-797, and involves a two-step process: (i) activation of amine groups (either primary or secondary) on the surface of the latex particles with 5% v/v glutaraldehyde in water, followed by (ii) covalent attachment of HSA via an overnight incubation in a low ionic strength phosphate buffer (see FIG. 16).

The glutaraldehyde activation approach provides the following benefits:
Excellent discrimination between HSA-containing samples and non-HSA-containing samples in a competitive assay format (see FIG. 10)
Good release from sprayed accretion pad into lysis buffer
Maintains particle stability in storage buffer at 2-8 degrees Celsius
Lowest cost approach compared with many other options (e.g. inexpensive linker, less protein excess required, >90% yield of original latex stock)
Low interaction with intended sample matrix, i.e., human nasal swabs

### Co-Coupled HSA+IgY Latex Particles

One potential issue with a competitive assay approach is the lack of positive feedback provided to the user when a negative test result occurs. It is common practice in lateral flow assays to include an internal control downstream from the capture line of the main target analyte. This helps inform the user that the test has been manufactured correctly, that the detector particles are functional and that the test has run to completion. This commonly involves an anti-species capture antibody which directly binds detector particles conjugated with antibodies from the corresponding host species. For example, an anti-mouse capture antibody would be a suitable internal control in a lateral flow assay which uses mouse antibodies conjugated to their detector particles.

In the Home Flu Test, an anti-mouse capture antibody would not be a suitable internal control for two reasons: (i) the fluorescent detector particles contain mouse antibodies which would compete with internal control particles, and (ii) mouse serum is added to the test as a blocking agent and this would rapidly saturate the anti-mouse capture line.

Instead, an internal control based on chicken IgY antibody has been developed (see FIG. 18). Chicken IgY has several advantages: (i) it is readily produced and extracted from chicken eggs in high yield, (ii) it is structurally different from mammalian IgG antibodies and has thus has no cross-reactivity with known human interferants such as complement, rheumatic factors or Fc-receptors, and (iii) several anti-species capture antibodies raised against chicken IgY are commercially available, e.g. goat anti-chicken IgY, donkey F(ab′)₂ anti-chicken IgY, rabbit F(ab′)₂ anti-chicken IgY and monoclonal mouse anti-chicken IgY.

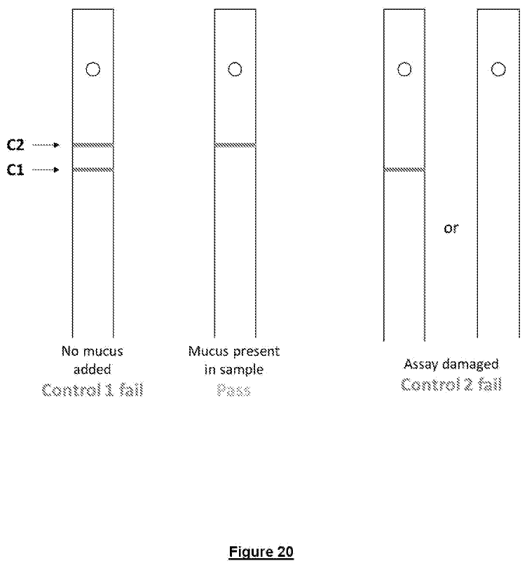

Another advantage of having a second control assay is less obvious. If both proteins (i.e. HSA and chicken IgY) are co-coupled onto the same batch of latex particles, then every particle is capable of binding to either control line. This may be accomplished by mixing both proteins prior to incubation with the glutaraldehyde-activated latex particles. Two scenarios can now occur (see FIG. 19):
1) In the absence of free HSA in the test sample, the protein-conjugated detector particles bind to either the anti-HSA (C1) or anti-chicken IgY (C2) capture lines in a constant ratio. This ratio is independent of how many particles were released from the accretion pad or the total volume applied to the test.
2) In the presence of free HSA in the test sample, there is a large change in this ratio due to much less binding at the anti-HSA capture line and slightly higher binding at the anti-chicken IgY capture line (due to less particles bound at C1 and hence available for binding at C2).

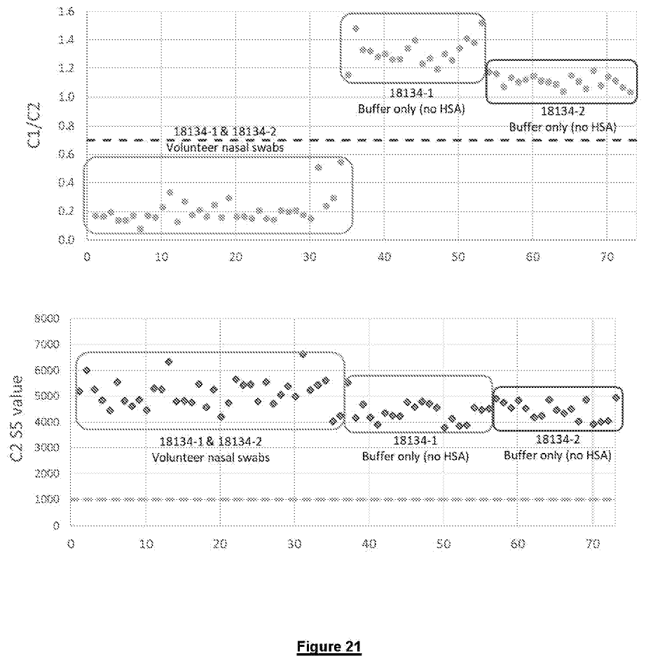

Therefore, a valid test result is obtained only when the ratio of C1/C2 is below a threshold value and the C2 value is above a threshold value, i.e. a sufficient number of functional particles are detected at C2 (see FIG. 20 and FIG. 21).

### Example 2

Each Low Positive FluA or Low Positive FluB sample was prepared by mixing 50 μL of an influenza nucleoprotein solution (diluted in PBS) and 450 μL of lysis buffer in a micro-vial. The micro-vial also contained an absorbent pad onto which the following particles were dried: (i) fluorescent Supernova particles co-coupled to anti-influenza A and B nucleoprotein, and (ii) blue-dyed latex particles co-coupled to HSA and chicken IgY. 125 682 L of this mixture was then added to the sample port of a HFT test device.

Buffer-only samples were similarly prepared by adding 50 μL of PBS and 450 μL of lysis buffer in a micro-vial.

Volunteer nasal swab samples were similarly prepared by swabbing a healthy volunteer with a nasal swab and immersing the swab tip into 450 μL of lysis buffer.

Calculated values for the fluorescent immunoassay (S5 value), internal control (Control value) and final test result are summarised in Table 1.

As is apparent from Table 1, all thirty-six samples gave the expected result for both the fluorescent immunoassay and internal control assay. The presence of both fluorescent Supernova particles and latex particles in the same sample did not appear to affect either assay.

TABLE 1
Dataset of contrived low positive (FluA or FluB) samples,
buffer-only samples and volunteer nasal swab samples.

| | | Test result as |
|---|---|---|
| Sample Name | Test result | expected? |
| LPA 01 | FluA Positive* | Y |
| LPA 02 | FluA Positive | Y |
| LPA 03 | FluA Positive | Y |
| LPA 04 | FluA Positive | Y |
| LPA 05 | FluA Positive | Y |
| LPA 06 | FluA Positive | Y |
| LPA 07 | FluA Positive | Y |
| LPA 08 | FluA Positive | Y |
| LPA 09 | FluA Positive | Y |
| LPA 10 | FluA Positive | Y |
| Blank 01 | Test error: error code 20^{#} | Y |
| Blank 02 | Test error: error code 20 | Y |
| Blank 03 | Test error: error code 20 \| | Y |
| Blank 04 | Test error: error code 20 | Y |
| Blank 05 | Test error: error code 20 | Y |
| LPB 01 | FluB Positive | Y |
| LPB 02 | FluB Positive | Y |
| LPB 03 | FluB Positive | Y |
| LPB 04 | FluB Positive | Y |
| LPB 05 | FluB Positive | Y |
| LPB 06 | FluB Positive | Y |
| LPB 07 | FluB Positive | Y |
| LPB 08 | FluB Positive | Y |
| LPB 09 | FluB Positive | Y |
| LPB 10 | FluB Positive | Y |
| Volunteer swab 01 | Negative | Y |
| Volunteer swab 02 | Negative | Y |
| Volunteer swab 03 | Negative | Y |
| Volunteer swab 04 | Negative | Y |
| Volunteer swab 05 | Negative | Y |
| Volunteer swab 06 | Negative | Y |
| Volunteer swab 07 | Negative | Y |
| Volunteer swab 08 | Negative | Y |
| Volunteer swab 09 | Negative | Y |
| Volunteer swab 10 | Negative | Y |

1. A lateral flow test strip comprising:
a) a first mobilisable labelled species capable of binding to a first control analyte;
b) a first control portion comprising a first immobilised capture reagent;
wherein the first immobilised capture reagent mimics at least one binding property of the first control analyte such that the first immobilised capture reagent is capable of binding to the mobilisable labelled species.

2. A lateral flow test strip comprising:
a) a first mobilisable labelled species mimicking at least one binding property of a first control analyte;
b) a first control portion comprising a first immobilised capture reagent;
wherein the first immobilised capture reagent is capable of binding to the mobilisable labelled species or to the first control analyte.

3. The lateral flow test strip of claim 1 or claim 2, further comprising:
c) a second mobilisable labelled species; and
d) a second control portion comprising a second immobilised capture reagent,
wherein the second immobilised capture reagent is capable of binding to the second mobilisable labelled species.

4. The lateral flow test strip according to claim 1, wherein in use, in the absence of the first control analyte in a test sample, the first mobilisable labelled species binds to the first immobilised capture reagent.

5. The lateral flow test strip according to claim 1, wherein in use, in the presence of the first control analyte in a test sample, the first mobilisable labelled species binds to the first immobilised capture reagent at a reduced level compared to the level of binding in the absence of the first control analyte.

6. The lateral flow test strip according to claim 1, wherein the first control analyte is typically present in a test sample.

7. A lateral flow test strip comprising:
a) a mobilisable labelled species which is bound to a first control analyte and a second control analyte;
b) a first control portion comprising a first immobilised capture reagent, wherein the first immobilised capture reagent is configured to specifically bind to the first control analyte;
c) and a second control portion comprising a second immobilised capture reagent, wherein the second immobilised capture reagent is configured to specifically bind to the second control analyte;
wherein the first control analyte is an analyte which is typically present in a test sample and wherein the second control analyte is an analyte not typically present in the test sample.

8. The lateral flow test strip according to claim 7, wherein in use, in the absence of the first control analyte in a test sample, the amount of the mobilisable labelled species immobilised at the first control portion is about equal to the amount of the mobilisable labelled species immobilised at the second control portion.

9. The lateral flow test strip according to claim 8, wherein the amount of the mobilisable labelled species immobilised at the first control portion and the amount of the mobilisable labelled species immobilised at the second control portion is present in about a 1:1 ratio to about a 2:1 ratio.

10. The lateral flow test strip according to claim 7, wherein in use, in the presence of the first control analyte in a test sample, the amount of the mobilisable labelled species immobilised at the first control portion is less than the amount of mobilisable labelled species immobilised at the second control portion.

11. The lateral flow test strip according to claim 10, wherein the amount of the mobilisable labelled species immobilised at the first control portion and the amount of the mobilisable labelled species immobilised at the second control portion is present in less than a 1:1 ratio.

12. The lateral flow test strip according to claim 1, wherein the labelled species is a latex particle, colloidal gold, magnetic particle or a nanoparticle aggregate.

13. The lateral flow test strip according to claim 1, wherein the labelled species is a glutaraldehyde-activated latex particle.

14. The lateral flow test strip according to claim 1, wherein the first control analyte is human serum albumin (HSA).

15. The lateral flow test strip according to claim 1, wherein the first immobilised capture reagent is an anti-human serum albumin antibody.

16. The lateral flow test strip according to claim 3, wherein the second control analyte is chicken IgY.

17. The lateral flow test strip according to claim 3, wherein the second immobilised capture reagent is an anti-chicken IgY antibody.

18. The lateral flow test strip according to claim 1, wherein the test sample is a biological sample.

19. The lateral flow test strip according to claim 1, wherein the test sample is a human sample.

20. The lateral flow test strip according to claim 18, wherein the test sample is a mucus sample.

21. The lateral flow test strip according to claim 18, wherein the test sample is a blood sample.

22. A device comprising a lateral flow test strip according to claim 1.
